(19) United States
(12) Patent Application Publication
(10) Pub. No.: US 2016/0346389 A1
(43) Pub. Date: Dec. 1, 2016

(54) MODIFIED PARAMAGNETIC NANOPARTICLES FOR TARGETED DELIVERY OF THERAPEUTICS AND METHODS THEREOF

(71) Applicant: ALBERT EINSTEIN COLLEGE OF MEDICINE INC., Bronx, NY (US)

(72) Inventors: Joel FRIEDMAN, South Orange, NJ (US); Navati MAHANTESH, Bronx, NY (US)

(21) Appl. No.: 15/021,333

(22) PCT Filed: Sep. 12, 2014
(86) PCT No.: PCT/US2014/055437

§ 371 Date: Mar. 11, 2016

Related U.S. Application Data
(60) Provisional application No. 61/876,923, filed on Sep. 12, 2013.
(60) Provisional application No. 62/047,242, filed on Sep. 8, 2014.

(51) Int. Cl. A61K 41/00; A61K 31/704; A61K 31/12; A61K 47/48; A61K 38/16; A61K 9/51; A61K 9/50; A61K 31/713; A61K 9/19; A61K 31/407

(52) U.S. Cl. CPC A61K 41/00; A61K 9/19; A61K 31/704; A61K 31/12; A61K 31/407; A61K 38/16; A61K 9/5169; A61K 9/5192; A61K 9/5146; A61K 9/5015; A61K 31/713; A61K 47/4893

(57) ABSTRACT

Described herein is a method of making modified paramagnetic nanoparticles with improved therapeutic loading efficiency and enhanced circulation properties. The method comprises coating a paramagnetic nanoparticle (PMNP) with a hydrophobic coating comprising lipophilic drug and a polymer. Also described herein is a PMNP, and a composition comprising PMNP. In certain embodiment, the PMNP have improved permeability through the blood brain barrier. Also described herein is a method of using the PMNP for the treatment of diseases. In certain embodiments, the method of treatment is a combination therapy. Described herein are imaging of therapeutic delivery of PMNP and diagnostic methods using the PMNP. Also described herein is a diagnostic kit that comprises the PMNP. The invention provides compositions comprising a paramagnetic nanoparticle having an external coating comprising a small organic molecule, a polymer, a blood protein, oleic acid, a lipophilic pharmaceutical or an allosteric effector of hemoglobin, as well as methods of making thereof, and use thereof in treatment and imaging.

### CROSS-REFERENCE TO RELATED APPLICATIONS

This application claims the benefit under 35 U.S.C. §119 §of U.S. Provisional Patent Application No. 61/876,923, filed Sep. 12, 2013 and U.S. Provisional Patent Application No. 62/047,242, filed Sep. 8, 2014, which are hereby incorporated by reference in their entireties.

### 1. INTRODUCTION

Described herein is a method of making modified paramagnetic nanoparticles with improved therapeutic loading efficiency and enhanced circulation properties. The method comprises coating a paramagnetic nanoparticle (PMNP) with a hydrophobic first layer comprising lipophilic drug and a polymer. Also described herein is a PMNP, and a composition comprising PMNP. In certain embodiment, the PMNP have improved permeability through the blood brain barrier. Also described herein is a method of using the PMNP for the treatment of diseases. In certain embodiments, the method of treatment is a combination therapy. Described herein are imaging of therapeutic delivery of PMNP and diagnostic methods using the PMNP. Also described herein is a diagnostic kit that comprises the PMNP. The invention provides compositions comprising a paramagnetic nanoparticle having an external coating comprising a small organic molecule, a polymer, a blood protein, oleic acid, a lipophilic pharmaceutical or an allosteric effector of hemoglobin, as well as methods of making thereof, and use thereof in treatment and imaging.

### 2. BACKGROUND OF THE INVENTION

There are several nanoparticle platforms that seek to achieve one or more of the objectives of high local concentrations of drugs, delivery vehicle for drugs with poor bioavailabilty issues due to poor solubility, targeted delivery which increases efficacy and minimized systemic side effects, multiple delivery routes, delivery of drug combinations, enhancing chemotherapeutic efficacy and reducing or preventing drug resistance and limiting consequences of radiation treatment. Most strategies for tissue targeting of nanoparticles are based on attachment of targeting molecules (e.g. antibodies, peptides, aptamers) on the surface of drug loaded nanoparticles. This approach has proven more challenging than anticipated in that circulating nanoparticles typically accumulate molecules such as proteins as they circulate which limits targeting efficacy. Previously available PMNPs have low loading capacity for therapeutic agents. In most instances the building of a therapeutically significant localized population of nanoparticles via this strategy requires overcoming the challenging requirement that the nanoparticles circulate for many hours without loss of their deliverables. Circulating paramagnetic nanoparticles can be localized in tissues via the application of an external field but there is the necessity of developing coatings that increase the loading capacity of therapeutically relevant molecules, improved control of the release of therapeutics at target cells, and for enhancing circulation properties.

Previous platforms used to coat PMNPs with oleic acid consisted of preparative methods that required mixing the oleic acid with reagents as the particles were being formed. These platforms describe protocols for coating PMNPs that are typically only a few nanometers in diameter which limits the drug carrying capacity of the nanoparticle. There is a need for a new platform which allows the coating and derivatization of synthesized PMNPs of all sizes.

The present invention provides coating strategies for paramagnetic nanoparticles for: i) targeted delivery of therapeutics including chemotherapeutics, anti-inflammatories such as curcumin, lipohilic molecules in general, curcumin, nitric oxide, plasmids and red blood cell effective allosteric effectors of hemoglobin; and ii) enhanced circulation properties and further surface modification with respect to tissue specific targeting via PEGylation.

### 3. SUMMARY OF THE INVENTION

Described herein is a rapid and efficient method of making paramagnetic nanoparticles of any dimension (microns to a few nanometers) that have partial or complete surface modifications, such as a coating, which provides high drug capacity loading, high dispersibility, stability in a nonpolar organic solvent or an aqueous solution with high yield without the loss of PMNP, improved circulation in the blood stream and improved penetration across blood brain barrier. In certain embodiments, the PMNP are coated with fatty acids, including oleic acid, conjugated linoleic acid and nitro-fatty acids and PEG chains of varied sizes with or without derivatizations that allow for surface attachment of additional molecules such as targeting molecules and imaging agents. In certain embodiment, the disclosure provides the ability to coat larger nanoparticles which allows for greater drug loading. In certain embodiments, using gadolinium oxide nanoparticles as a core results in more efficient coatings compared to protocols that first coat the gadolinium hydroxide derivative followed by heating to make the paramagnetic oxide. In certain embodiment, a gadolinium oxide core comprises a first coating. In certain embodiment, the method does not comprise applying heat to gadolinium hydroxide to form the gadolinium oxide core. In certain embodiment, the method does not involve the coating of gadolinium hydroxide derivative followed by applying heat to gadolinium hydroxide to form gadolinium oxide.

This new approach allows for very rapid and enhanced (amount delivered per unit time) drug delivery to tissues targeted using an external magnetic field. The present disclosure demonstrated major enhanced drug efficacy with respect to tumor killing with no systemic toxicity. The present disclosure also shows efficient drug delivery to brain tumors that greatly exceeds previous capabilities. Also described herein is the use of a magnetic field enhanced blood brain barrier (“BBB”) crossing at a level comparable to the best BBB crossing reagents but with the dramatic advantage of much higher levels of drug delivery (due to the high drug loading capacity of the large coated nanoparticles).

In certain embodiments, localization within certain tissues is also possible utilizing the EPR effect (enhanced penetration and retention effect) that arises from leaky blood vessels associated with inflamed tissues (many cancers) that allow for the trapping of circulating nanoparticles.

The present disclosure demonstrated glioblastoma targeting with evidence of tumor shrinkage through the targeted delivery of very high levels of curcumin (loaded on the OA-coating of 100 nm PMNPs. In certain embodiments, the concentration of circumin on OA-PMNP is 10-15 μg, 15-20 μg, 20-25 μg, 25-30 μg, 30-35 μg, 35-40 μg, 22-44 μg/mg of PMNPs.

The present disclosure provides a platform that creates large drug loaded PMNPs that are rapidly taken up by many kinds of cells most notably tumor cells.

Provided herein is a method of making modified paramagnetic nanoparticles having improved loading of therapeutics and enhanced circulation properties. In certain embodiments, the modified PMNPs enhance the binding of lipophilic drugs by 5-10, 10-20, 20-30, 30-40, 40-50, 50-60, 60-70 folds. In certain embodiments, the modified PMNPs have the size of, 40-50, 50-60, 60-70, 70-80, 80-90, 90-100, 100-150, 150-200, 200-250, 250-300 nm.

The method comprising coating a paramagnetic nanoparticle (PMNP) with a hydrophobic coating comprising lipophilic drug and a polymer. In certain embodiments, the method comprises making paramagnetic nanoparticles coated with: i) fatty acids; ii) fatty acids and a polymer, such as polyethylene glycol (PEG), optionally with the following: lipophilic drugs including Adriamycin and other chemotherapeutics, curcumin, melanin, plasmids, siRNA, allosteric effectors of hemoglobin, imaging agents, or combination thereof. In certain embodiments, the lipophilic drug is Adriamycin (ADM), curcumin, taxol, allosteric effector (L35), anti-inflammatories, siRNA, plasmids, nitro fatty acids or a combination thereof. In certain embodiments, the polymer is PEG. In certain embodiments, the polymer is linked to a targeting molecule. In certain embodiments, the method of making a drug-loaded albumin-coated paramagnetic nanoparticle composition comprising admixing and sonicating (a) a solution of paramagnetic nanoparticles in an ethanol in methanol solution with (b) a solution of the drug to be loaded in methanol, and adding an albumin in aqueous solution to the admixture of (a) and (b) so as to make the drug-loaded albumin-coated paramagnetic nanoparticle composition. In certain embodiments, the plasmid is pVAX-hSL01.

In one embodiment, provided herein is a method of making modified PMNP comprising the steps of: (i) adding fatty acid to PMNP core to form a mixture; (ii) Sonicating the mixture; (iii) Spinning the sonicated mixture and washing in deionized water; (iv) Drying and lyophilizing the washed mixture to form a powder; (v) mixing the lyophilized powder with a non-aqueous concentrated solution of a therapeutic agent to form a mixture; (vi) sonicating the mixture from step (v); (vii) spinning the sonicated mixture and washing in deionized water.

In one embodiment, provided herein is a method of making a modified PMNP comprising the steps of: (i) adding fatty acid to PMNP core to form a mixture; (ii) Sonicating the mixture; (iii) Spinning the sonicated mixture and washing in deionized water to form an aqueous suspension; (iv) mixing the aqueous suspension with a non-aqueous concentrated solution comprising a therapeutic agent; (v) sonicating the mixture from step (vi); (vi) removing the non-aqueous solvent. In certain embodiments, the therapeutic agent is Adriamycin, taxol, curcumin, dasationib, melanin, allosteric effector, albumin, plasmid, siRNA or a combination thereof.

In certain embodiment, the method further comprises adding methoxy PEG-DSPE, fluorescence-labeled PEG-DSPE, or a derivatized PEG-DSP to the sonicated mixture. In certain embodiments, the PEG-DSPE comprises a reactive species including maleimide, amine, thiol or a combination thereof. In certain embodiment, the reactive species is attached to a fluorophore, PET imaging agent, peptide, antibody, aptamer, contrasting agent or a combination thereof. In certain embodiment, the peptide is CXCR4 antagonistic peptide.

In one embodiment, provided herein is a method of making a drug-loaded albumin-coated paramagnetic nanoparticle (alb-PMNP) comprising the steps of: (i) mixing an ethanol in methanol solution comprising PMNP core with a methanol solution comprising a therapeutic agent to form a mixture; (ii) sonicating the mixture; (iii) adding an aqueous solution comprising albumin to the sonicated mixture. In certain embodiments, the method further comprises the step of: (i) adding methoxy PEG-DSPE, fluorescence-labeled PEG-DSPE, or a derivatized PEG-DSP to the sonicated mixture in step (iii). In certain embodiments, the PEG-DSPE comprises a reactive species including maleimide, amine, thiol or a combination thereof. In certain embodiments, the reactive species is attached to a fluorophore, PET imaging agent, peptide, antibody, aptamer, contrasting agent or a combination thereof. In certain embodiments, the peptide is CXCR4 antagonistic peptide.

In one embodiment, provided herein is a method of making modified PMNP comprising the steps of: (i) adding 3-mercaptopropyl-trimethoxysilane (3MPTS) or (N-(2-Aminoethoxyl)-11-Aminoundec-yl trimethoxysilane) (APTS) to a solution containing PMNP core in deionized water to form a mixture; (ii) Sonicating the mixture in step (i); (iv) incubating the sonicated mixture at 4° C.; (v) washing the mixture in deionized water; and (vi) adding 4′-dithiodipyridine (4-PDS) to form a mixture. In certain embodiments, the method further comprises the step of adding 2-imminothiolane & mal-PEG-5K to the mixture of step (vi). In certain embodiment, the method further comprises the step of: (i) adding dithiothreitol (DTT) to the mixture of step (vi); (ii) treating the mixture with buffer saturated with pure NO gas. In certain embodiment, the PMNP core comprises substantially of Gd₂O₃ or iron oxide. In certain embodiments, the Gd₂O₃ is doped with europium or other lanthanides. In certain embodiments, the fatty acid is oleic acid.

In one embodiment, provided herein is a method of delivering the modified PMNP to a target location in a subject comprising: (i) administering to the subject an effective amount of the modified PMNP; and (ii) applying a magnetic field to the subject, such that the magnetic field is present at the target location at a strength sufficient to attract the modified PMNP.

In certain embodiment, the modified PMNP is administered systemically. In certain embodiment, the location of the modified PMNP is monitored using MRI. In certain embodiment, the modified PMNP comprises fluorophores.

In one embodiment, provided herein is a method of treating cancer in a subject comprising: (i) administering to the subject an effective amount of the modified PMNP; and (ii) applying a magnetic field to the subject at the location of the cancer, and wherein the magnetic field is at a strength sufficient to attract the modified PMNP to the cancer. In certain embodiment, the therapeutic agent is a chemotherapeutic drug, small organic molecule, cytotoxic drug, or a combination thereof. In certain embodiment, the cancer is pancreatic cancer, CNS cancer, bone cancer, hypoxic tumor. In certain embodiment, the subject is treated with a second cancer therapy, i.e, combination therapy.

In one embodiment, provided herein is a method of treating cancer in a subject comprising: (i) administering to the subject an effective amount of the modified PMNP; and (ii) applying a magnetic field to the subject at the location of the cancer, and wherein the magnetic field is at a strength sufficient to attract the modified PMNP to the cancer. In certain embodiment, the peptide binds to a cell-surface target.

In one embodiment, provided herein is a method of treating sickle cell disease in a subject comprises administering to the subject an effective amount of the modified PMNP, wherein the therapeutic agent is an allosteric effector.

In one embodiment, provided herein is a method of treating an inflammation in a subject comprising: (i) administering to the subject an effective amount of the modified PMNP; and (ii) applying a magnetic field to the subject at the location of the inflammation, and wherein the magnetic field is at a strength sufficient to attract the modified PMNP to the predetermined location. In certain embodiment, the inflammation is at a joint.

In one embodiment, provided herein is a method of treating or reducing a reperfusion injury or ischemia in a subject comprising: (i) administering to the subject an effective amount of the modified PMNP; and (ii) applying a magnetic field to the subject at the location of the reperfusion injury or ischemia, and wherein the magnetic field is at a strength sufficient to attract the modified PMNP to the reperfusion injury or ischemia.

In one embodiment, provided herein is a method of imaging a predetermined location in a subject comprising: (i) administering to the subject an effective amount of the modified PMNP; (ii) applying a magnetic field to the subject predetermined location at a strength sufficient to attract the modified PMNP to the predetermined location; and (iii) collecting an imaging signal from the predetermined location using an imaging device so as to thereby image the predetermined location.

In one embodiment, provided herein is a method of increasing oxygen levels in a target tissue in a subject having a disorder comprising: (i) administering to the subject an effective amount of the modified PMNP; and (ii) applying a magnetic field to the subject at the predetermined location where an increased oxygen level is desired, and wherein the magnetic field is at a strength sufficient to attract the modified PMNP to the predetermined location. In certain embodiment, the disorder is cancer, hypoxic tumor, sickle cell anemia, or local hypoxic conditions.

In one embodiment, provided herein is a modified paramagnetic nanoparticle (PMNP) comprising a PMNP core, which core comprises a coating, said coating comprising oleic acid, a fatty acid, albumin, or a combination thereof, said coating is dispersed therewith an allosteric effector of hemoglobin, curcumin, melanin, siRNA, plasmids, nitro fatty acids, adriamycin, taxol, or a combination thereof and wherein polymer PEG-DSPE are attached to the PMNP core. In certain embodiment, the allosteric effector of hemoglobin is, 2, 3-Bisphosphoglycerate (2, 3-BPG), Myo-inositol trispyrophosphate (ITPP), or a combination thereof. In certain embodiment, the coating comprises an albumin and wherein the coating is dispersed therewith curcumin, Adriamycin, taxol and wherein the polymer 1, 2-distearoyl-sn-glycero-3-phosphoethanolamine-N-[amino(polyethylene glycol)-2000 (PEG-DSPE) is attached to the PMNP core. In certain embodiment, the PEG-DSPE comprises a reactive species including maleimide, amine, thiol or a combination thereof. In certain embodiment, the reactive species is attached to fluorophores, PET imaging agents, peptides, antibodies, aptamers, contrasting agents or a combination thereof. In certain embodiment, the coating comprises oleic acid and wherein the coating is dispersed therewith curcumin, Adriamycin, taxol and wherein the polymer 1, 2-distearoyl-sn-glycero-3-phosphoethanolamine-N-[amino(polyethylene glycol)-2000 (PEG-DSPE) is attached to the PMNP core.

In certain embodiment, the PEG-DSPE comprises a reactive species including maleimide, amine, thiol or a combination thereof. In certain embodiment, the reactive species is attached to fluorophores, PET imaging agents, peptides, antibodies, aptamers, contrasting agents or a combination thereof.

Provided herein is a PMNP and a composition comprising a paramagnetic nanoparticle having an external coating comprising a small organic molecule, a polymer, a blood protein, oleic acid, a lipophilic pharmaceutical, an allosteric effector of hemoglobin or a combination thereof.

Also described herein is a method of using the PMNP for the treatment of diseases. Provided herein is a method of delivering small organic molecules, polymers, blood proteins, plasmids, siRNA, nitric oxide or nitric oxide-releasing compounds, oleic acid and other fatty acids including nitro-fatty acids, lipophilic pharmaceuticals, or allosteric effectors of hemoglobin, to a predetermined location in a subject comprising administering to the subject a composition as described herein, or a composition comprising nitric oxide-releasing paramagnetic nanoparticles or nitric oxide-releasing compound-releasing paramagnetic nanoparticles, and applying a magnetic field to the subject, such that the magnetic field is present in the predetermined location at a strength sufficient to attract an administered composition. In certain embodiments, the method of treatment is a combination therapy.

Also described is the development of paramagnetic nanoparticles that can deliver high therapeutic doses of nitric oxide to targeted tissues though the use of an external magnetic field. In certain embodiments, the PMNP are used to treat ischemic tissue with low tissue perfusion, enhance drug and oxygen delivery to poorly perfused tissue, treat cardiogenic shock, prevent ischemia reperfusion injury in targeted tissues, normalize the nitric oxide gradient in tumor vasculature and thus reestablish healthy vessels in tumors which limits tumor growth and metastasis while enhancing tumor oxygenation and drug delivery.

Also described herein is the development of a paramagnetic nanoparticle platform capable of enhancing oxygen tension in targeted tissues. In certain embodiments, the disclosed method obviates the need for costly use of hyperbaric chambers to treat localized areas requiring enhanced oxygenation and to treat cardiogenic shock.

Described herein is a theranostic method of combining diagnostic imaging and drug delivery using the PMNP. Also provided is a theranostic of combining diagnostic imaging and drug delivery at a predetermined location in a subject comprising administering to the subject the composition described herein that comprise an imaging agent and applying a magnetic field to the subject, such that the magnetic field is present in the predetermined location at a strength so as to attract the composition to a predetermined location, and collecting an imaging signal from the predetermined location and at the same time deliver a therapeutic to this same site.

In one embodiment, provided herein is a composition comprising a paramagnetic nanoparticle having an external coating comprising a small organic molecule, a polymer, a blood protein, oleic acid, a lipophilic pharmaceutical or an allosteric effector of hemoglobin.

In an embodiment, provided herein is a method of delivering a small organic molecule, a polymer, a blood protein, nitric oxide or nitric oxide-releasing compound, oleic acid, an oleic acid having admixed therewith a lipophilic pharmaceutical, or an allosteric effector of hemoglobin, to a predetermined location in a subject comprising administering to the subject the composition disclosed herein, or a composition comprising nitric oxide-releasing nanoparticles or nitric oxide-releasing compound-releasing nanoparticles, and applying a magnetic field to the subject, such that the magnetic field is present in the predetermined location at a strength sufficient to attract an administered paramagnetic nanoparticle composition.

In one embodiment, provided herein is a method of imaging a predetermined location in a subject comprising administering to the subject the composition described herein and applying a magnetic field to the subject, such that the magnetic field is present in the predetermined location at a strength so as to attract the composition to a predetermined location, and collecting an imaging signal from the predetermined location using an imaging device so as to thereby image the predetermined location.

In one embodiment, provided herein is a method of making a drug-loaded albumin-coated paramagnetic nanoparticle composition comprising admixing and sonicating (a) a solution of paramagnetic nanoparticles in an ethanol in methanol solution with (b) a solution of the drug to be loaded in methanol, and adding an albumin in aqueous solution to the admixture of (a) and (b) so as to make the drug-loaded albumin-coated paramagnetic nanoparticle composition.

Also described herein is a kit that comprises the PMNP.

### 3.1 Definitions

As used herein, the term “agent” refers to any molecule, compound, and/or substance for use in the prevention, treatment, management and/or diagnosis of a disease, including but not limited to cancer.

As used herein, the term “amount,” as used in the context of the amount of a particular cell population or cells, refers to the frequency, quantity, percentage, relative amount, or number of the particular cell population or cells.

As used herein, the term “bind” or “bind(s)” refers to any interaction, whether direct or indirect, that affects the specified receptor (target) or receptor (target) subunit.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. In yet other embodiments, the cancer is associated with a specific cancer antigen.

As used herein, the term “cancer cells” refers to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cytotoxin” or the phrase “cytotoxic agent” refers to a compound that exhibits an adverse effect on cell growth or viability. Included in this definition are compounds that kill cells or which impair them with respect to growth, longevity, or proliferative activity.

As used herein, the phrase “detectable agents” refers to any molecule, compound and/or substance that is detectable by any methodology available to one of skill in the art. Non-limiting examples of detectable agents include dyes, gas, metals, or radioisotopes.

As used herein, the terms “disorder” and “disease” are used interchangeably to refer to a pathological condition in a subject.

As used herein, the term “effective amount” refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

As used herein, the phrase “elderly human” refers to a human 65 years old or older, preferably 70 years old or older.

As used herein, the phrase “human adult” refers to a human 18 years of age or older.

As used herein, the phrase “human child” refers to a human between 24 months of age and 18 years of age.

As used herein, the phrase “human infant” refers to a human less than 24 months of age, preferably less than 12 months of age, less than 6 months of age, less than 3 months of age, less than 2 months of age, or less than 1 month of age.

As used herein, the term “in combination” in the context of the administration of a therapy to a subject refers to the use of more than one therapy (e.g., prophylactic and/or therapeutic). The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the terms “manage,” “managing,” and “management” in the context of the administration of a therapy to a subject refer to the beneficial effects that a subject derives from a therapy (e.g., a prophylactic or therapeutic agent) or a combination of therapies, while not resulting in a cure of cancer. In certain embodiments, a subject is administered one or more therapies (e.g., one or more prophylactic or therapeutic agents) to “manage” cancer so as to prevent the progression or worsening of the condition.

As used herein, the phrase “pharmaceutically acceptable” means approved by a regulatory agency of the federal or a state government, or listed in the United States Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a cancer or a symptom thereof in a subject resulting from the administration of a therapy (e.g., a prophylactic or therapeutic agent), or a combination of therapies (e.g., a combination of prophylactic or therapeutic agents). In some embodiments, such terms refer to one, two, three, or more results following the administration of one or more therapies: (1) a stabilization, reduction or elimination of the cancer stem cell population, (2) a stabilization, reduction or elimination in the cancer cell population, (3) an increase in response rate, (4) an increase in the length or duration of remission, (5) a decrease in the recurrence rate of cancer, (6) an increase in the time to recurrence of cancer, (7) an increase in the disease-free, relapse-free, progression-free, and/or overall survival of the patient, and (8) an amelioration of cancer-related symptoms and/or quality of life. In specific embodiments, such terms refer to a stabilization, reduction or elimination of the cancer stem cell population.

As used herein, the phrase “prophylactic agent” refers to any molecule, compound, and/or substance that is used for the purpose of preventing disease, including but not limited to cancer, autoimmune disease, or allergic disease. Examples of prophylactic agents include, but are not limited to, proteinaceous (such as immunoglobulins (e.g., multi-specific Igs, single chain Igs, Ig fragments, polyclonal antibodies and their fragments, monoclonal antibodies and their fragments and binding proteins), immunotoxins, chemospecific agents, chemotoxic agents (e.g., anti-cancer agents), and small molecule drugs.

As used herein, the term “prophylactically effective regimen” refers to an effective regimen for dosing, timing, frequency and duration of the administration of one or more therapies for the prevention of cancer or a symptom thereof. In a specific embodiment, the regimen achieves one, two, or three or more of the following results: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

As used herein, the term “protocol” refers to a regimen for dosing and timing of the administration of one or more agents and/or compositions for the prevention, treatment, and/or management of a disease or a symptom thereof. In certain embodiments, the term “protocol” refers to methods of patient care that are associated with the administration of an agent.

As used herein, the terms “purified” and “isolated” in the context of a compound or agent (including, e.g., proteinaceous agents such as antibodies) that is chemically synthesized refers to a compound or agent that is substantially free of chemical precursors or other chemicals when chemically synthesized. In a specific embodiment, the compound or agent is 60%, 65%, 75%, 80%, 85%, 90%, 95%, or 99% free (by dry weight) of other, different compounds or agents.

As used herein, the terms “purified” and “isolated” when used in the context of a compound or agent (including proteinaceous agents such as antibodies) that can be obtained from a natural source, e.g., cells, refers to a compound or agent that is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

As used herein, the phrase “small molecule(s)” and analogous terms include, but are not limited to, peptides, peptidomimetics, amino acids, amino acid analogs, polynucleotides, polynucleotide analogs, nucleotides, nucleotide analogs, and other organic and inorganic compounds (i.e., including hetero-organic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, organic or inorganic compounds having a molecular weight less than about 100 grams per mole, and salts, esters, and other pharmaceutically acceptable forms of such compounds.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “therapeutic agent” refers to any molecule, compound, and/or substance that is used for the purpose of treating and/or managing cancer. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, chemospecific agents, chemotoxic agents (e.g., anti-cancer agents), radiation, chemotherapy, anti-angiogenic agents, and small molecule drugs. Therapeutic agents may be a(n) anti-angiogenesis therapy, targeted therapy, radioimmunotherapy, small molecule therapy, biologic therapy, epigenetic therapy, toxin therapy, differentiation therapy, pro-drug activating enzyme therapy, antibody therapy, chemotherapy, radiation therapy, hormonal therapy, immunotherapy, or protein therapy.

As used herein, the term “therapeutically effective regimen” refers to a regimen for dosing, timing, frequency, and duration of the administration of one or more therapies for the treatment and/or management of cancer or a symptom thereof. In a specific embodiment, the regimen achieves one, two, three, or more of the following results: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

As used herein, the terms “therapies” and “therapy” can refer to any method(s), composition(s), and/or agent(s) that can be used in the treatment of a cancer or one or more symptoms thereof. In certain embodiments, the terms “therapy” and “therapies” refer to chemotherapy, radiation therapy, radioimmunotherapy, hormonal therapy, targeted therapy, toxin therapy, pro-drug activating enayme therapy, protein therapy, antibody therapy, small molecule therapy, epigenetic therapy, demethylation therapy, histone deacetylase inhibitor therapy, differentiation therapy, antiangiogenic therapy, biological therapy including immunotherapy and/or other therapies useful in the treatment of a cancer or one or more symptoms thereof.

As used herein, the terms “treat”, “treatment”, and “treating” in the context of the administration of a therapy to a subject refer to the reduction or inhibition of the progression and/or duration of cancer, the reduction or amelioration of the severity of cancer, and/or the amelioration of one or more symptoms thereof resulting from the administration of one or more therapies. In a specific embodiment, a patient that is at a high risk for developing cancer is treated. In specific embodiments, such terms refer to one, two, or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. In certain embodiments, such terms refer to a stabilization or reduction in the cancer cell population. In some embodiments, such terms refer to a stabilization or reduction in the growth of cancer cells. In some embodiments, such terms refer to a stabilization or reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

Concentrations, amounts, cell counts, percentages, and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. BRIEF DESCRIPTION OF THE DRAWINGS

FIG. 1. Schematic diagram of one embodiment of a drug loaded paramagnetic nanoparticle (PMNP) described herein with attached derivatized (or derivatized) PEG of varying size. The x on the PEG chain represents the specific derivative that allows for attachment of any of several possible cell and tissue specific targeting molecules. PEG increases the circulation time, enhances crossing of the blood brain barrier, limits aggregation, increases localization in tumors, and allows for attachment of a wide variety of molecules (targeting as well as imaging). Also shown are spheres indicating nitric oxide loading as described herein.

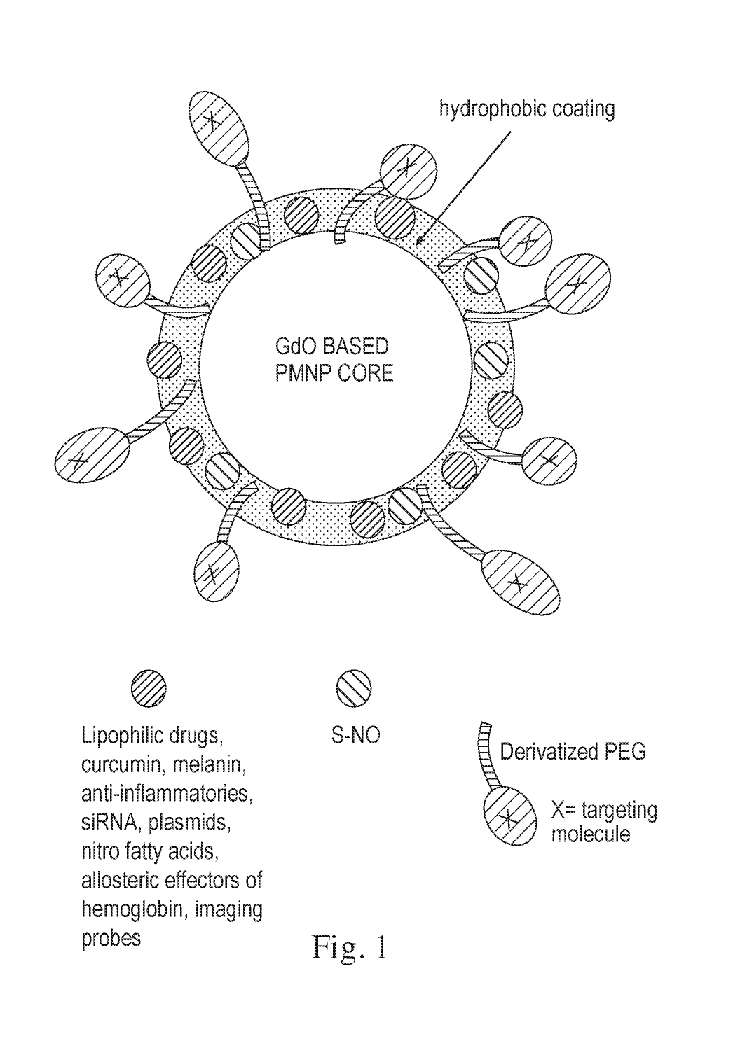

FIGS. 2(A-F). PMNP's (OA-PMNP (˜100 nm) with PEG) can be localized in healthy tissues (brain and dorsal skin) subsequent to IV administration followed by application of an external magnetic field. The figure shows results obtained using two physical techniques to detect paramagnetism in tissues. The shown heating with time is the result of an oscillating field inducing heating from the PMNPs. Both studies show that the application of an external magnetic field results in an enhanced and persistent accumulation of PMNP in the tissues to which the magnetic field is applied. The results are consistent with imaging results where the application of an external magnetic field produces enhanced fluorescence in the tissues surrounding the observed blood vessel indicating that the magnetic field can cause extravasation of the PMNPs even in normal “non-leaky” vessels. The results also indicate that these PMNPs can pass through the blood brain barrier and localize in brains of healthy animals.

FIGS. 3(A-D). FIG. 3A shows all of the tumors with minimal enhancement. By applying a magnet field for 30 minutes at hind end (blue arrows), the enhanced image of hind-end tumor is observed (3B). The enhanced image of the tumor persists for at least several hours. FIG. 3C shows another mouse with human breast xenographs. The PMNPs are concentrated at site of a lower abdominal mammary tumor (FIG. 3C) using an external magnetic field placed over the tumor for 30 minutes. The contrast increased significantly (FIG. 3D) subsequent to treatment with the magnet field. Imaging was performed over several hours without taking the mouse out of MRI machine thereby maintaining all of the imaging parameters. Enhanced images of the magnetic field targeted tumor site were observed as well as in the bladder which demonstrates efficient excretion of PMNPs that are not attached to tumor.

FIGS. 4(A-B). Compares the MRI contrast data between MAGNEVIST and gadolinium oxide-based PMNPs. The contrast for the MAGNEVIST is short lived whereas the contrast for the magnet targeted PMNP persists for hours and yields contrast comparable to MAGNEVIST.

FIG. 5. Viability test for three different types of cells, fibroblasts from normal mice and two cancer cell lines, treated with various concentrations of curcumin loaded OA-PMNP (gadolinium oxide-based) nanoparticle (n+c) as well as several controls including uncoated curcumin (c), carrier DMSO (D) and uncoated OA-PMNP (n). These cells were incubated for 24 hrs in a 96-well culture dish and then the amount of viable cells in each well was measured using MTT test. Higher OD in the graph indicates higher density of viable cells. The results indicate efficacy for the curcumin loaded OA-PMNPs with respect to killing of tumor cells.

FIG. 6. 100 μL, injection of curcumin loaded OA-PMNPs was given to two mice each with breast cancer xenographs (MDA-MB-436). In one mouse, an obvious tumor was exposed to a magnetic field for 45 minutes whereas no magnetic field was placed on the other mouse. A significant difference in the tumor-growth rates were observed over 6 days.

FIGS. 7(A-C). Site-specific delivery of magnetic particles (PMNP) detected by MRI. A-B. A PyMT mouse carrying multiple mammary tumors was tail vein injected with PMNP and an external magnetic field placed on the lower abdominal mammary tumor for 30 minutes. Imaging was performed continuously for 3 h (2 h images displayed). Note the increased intensity at the abdominal tumor and in the bladder (A) compared to background signals obtained at a control thoracic tumor not exposed to the magnetic field (B). Only background signals were observed at tumors in uninjected mice (C).

FIG. 8 Anti-tumor effect of IV infused Adriamycin loaded PMNP (ADM-PMNP) plus magnetic field treatment on xenografted human breast cancer. Human breast cancer was s.c. xenografted in a female nude mouse. The mouse was injected with 1 mg/kg ADM-PMNP and the tumor was treated with magnetic field for 30 minutes following each injection The mouse was imaged before and after the treatment, the luminescent signals were quantitatively analyzed for change of tumor size. At the end of the treatment, there was a >80% decrease of luminescent signaling in the tumor. The signal recovered partially 3 days after the treatment was ended. Treatment with a comparable amount of free Adriamycin was relatively ineffective.

FIGS. 9(A-B). ADM-PMNP+magnetic field exposure promotes tumor necrosis. (A) Correlated to the growth inhibition of xenografted human breast cancer in mice treated with both ADM-PMNP and magnetic field. (B) The necrosis was confirmed by histological analysis. Large necrosis sites were found in these treated tumors (top left, arrows) that was not seen in tumors from mice injected with ADM-free PMNP and treated with a magnetic field (top, middle).

FIGS. 10A-10D. Anti-tumor activity of ADM-PMNP for human prostate cancer. A nude mice xenografted with a human prostate (PC3) and breast cancer (M436) was treated with 1 mg/kg BW of ADM-PMNP (i.v. injection every two days for 3 injections). An external magnetic field was applied on the prostate tumor following ADM-PMNP administration. (A) Tumor growth in ADMP-PMNP/magnetic field exposed tumor was inhibited compared to untreated controls. (B) A large necrosis was observed in magnetic field exposed tumor, but not in human breast tumor adjacent to the prostate tumor (C), or PC tumor from a mouse without any treatment (D).

FIGS. 11(A-B). Systemic effect of the treatment. (A) Representative H&E staining of tissue sections from ADM-PMNP injected mice (1 mg/kg BW)(A). (B) inflammation and hyperplasia were found in colon from mice treated with free ADM (B-left) but not in mice injected with the same dose of ADM-PMNP (B-right). A more recent histology/pathology study in which ADM-PMNPs were injected into a larger series of mice over a three week period (every third day) and all organs examined in detail by a pathologist revealed no evidence of any tissue/organ damage.

FIGS. 12(A-B): ADM-PMNP+magnetic field exposed inhibits tumor outgrowth in bone. Bioluminescent imaging prior to treatment to the right thigh as exposed to a magnetic field following ADM-PMNP treatment (1 mg/kg) of a nude mouse 3 wk after cardiac inoculation of BoM-1833 breast cancer cells. Bioluminescent images were taken of the mouse before and after a series of 3 treatments at 2d intervals. (A). Histology of the right femur showing metastasis in the bone marrow cavity (arrow). (B). Quantitation of total luminescence in the indicated tumors (labeled as in panel C) before and after the ADM-PMNP treatment (arrows indicate times of treatment).

FIG. 13: Graph showing comparison of the efficacy with which different nanoparticles transit a membrane that is considered to be an effective model for the blood brain barrier. The results show that the PEGylated oleic acid coated PMNPs (labeled PMNP-PEG in the figure) are effective in crossing the BBB relative to silica hydrogel based nanoparticles (labeled as sol-gel) and that the rate is enhance with the addition of the external magnetic field. The level and rate of crossing the model BBB barrier for the PMNP-PEG is comparable to the values observed to materials with known high rates of crossing.

FIG. 14. Schematic for an experiment to evaluate the efficacy of nitric oxide (NO) releasing PMNPs with and without magnetic targeting with respect to reversing the physiological and pathological consequences of ischemia reperfusion injury (see FIGS. 20 and 21). The figure shows the optical skin flap window that allows for the monitoring of physiological parameters during the phases of the study.

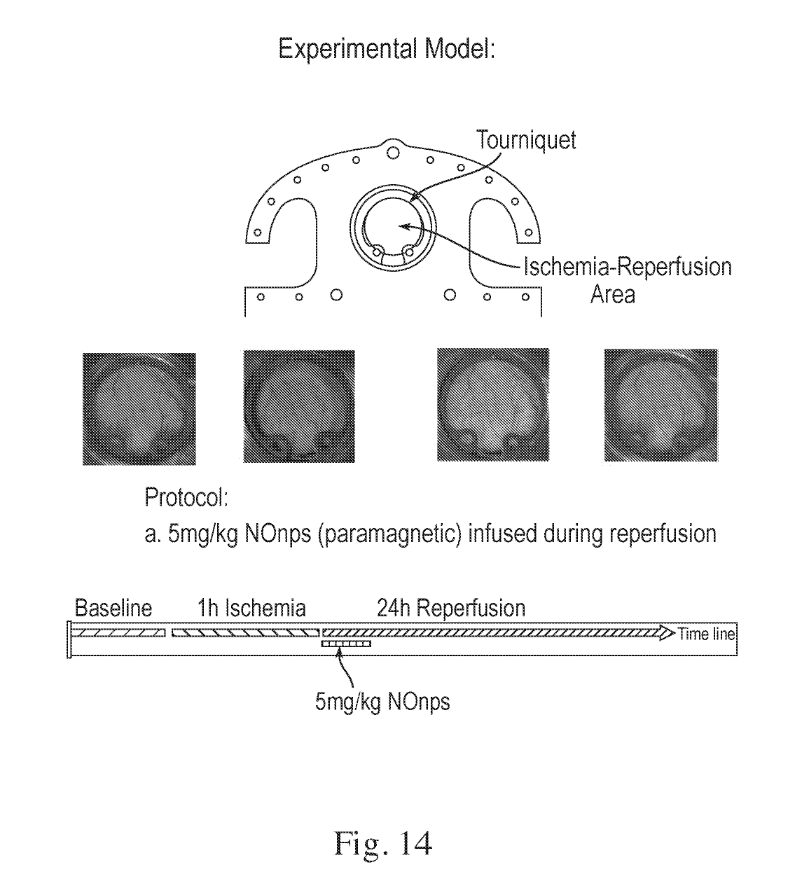

FIG. 15. Graph showing mean arterial pressure with NO-paramagnetic nanoparticles infused during reperfusion (systemic hypotension).

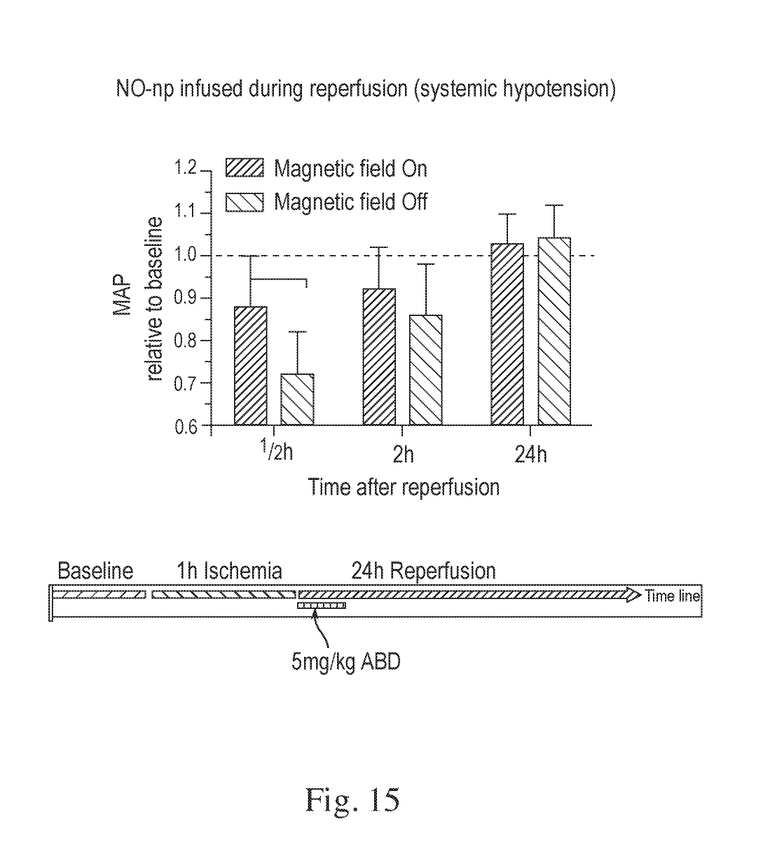

FIGS. 16(A-D). Graphs showing effect of NO-paramagnetic nanoparticles infusion in reperfusaion on arteriolar and venular flow and vessel diameter.

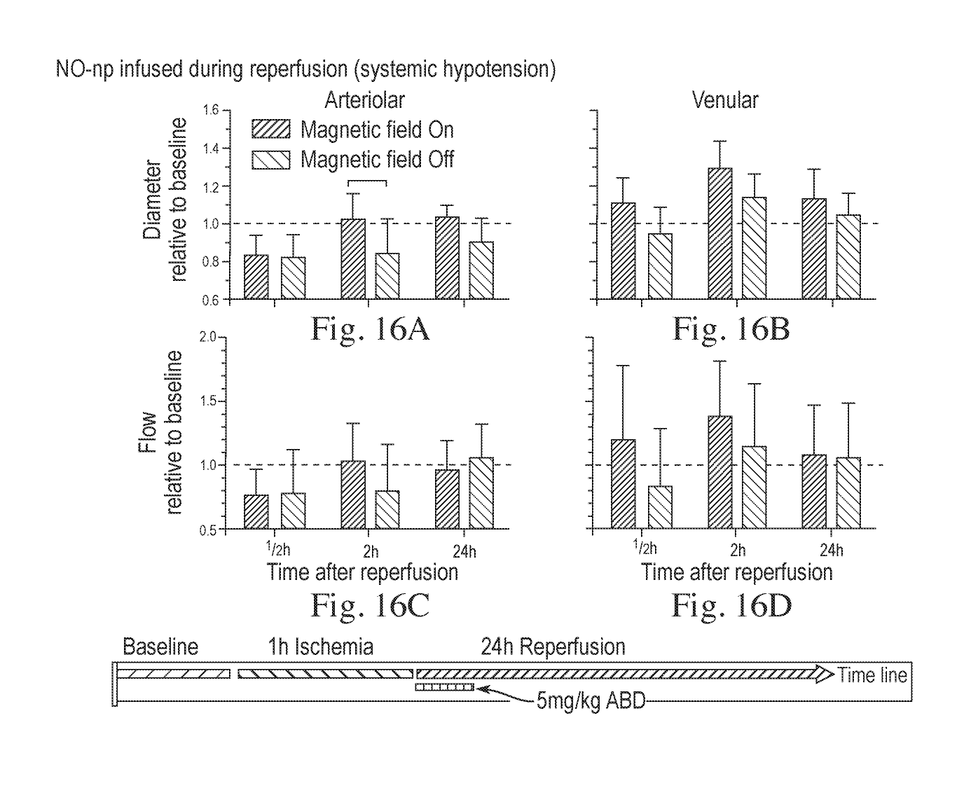

FIGS. 17(A-B). The figure shows results indicating that magnetic targeting of NO releasing PMNPs enhances functional capillary density (FCD) recovery in an iscehemia reperfusion (IR) injury model and limits IR induced inflammation as reflected in leucocyte immobilization (as well as leucocyte rolling—not shown). FCD is considered a major indicator of tissue recovery.

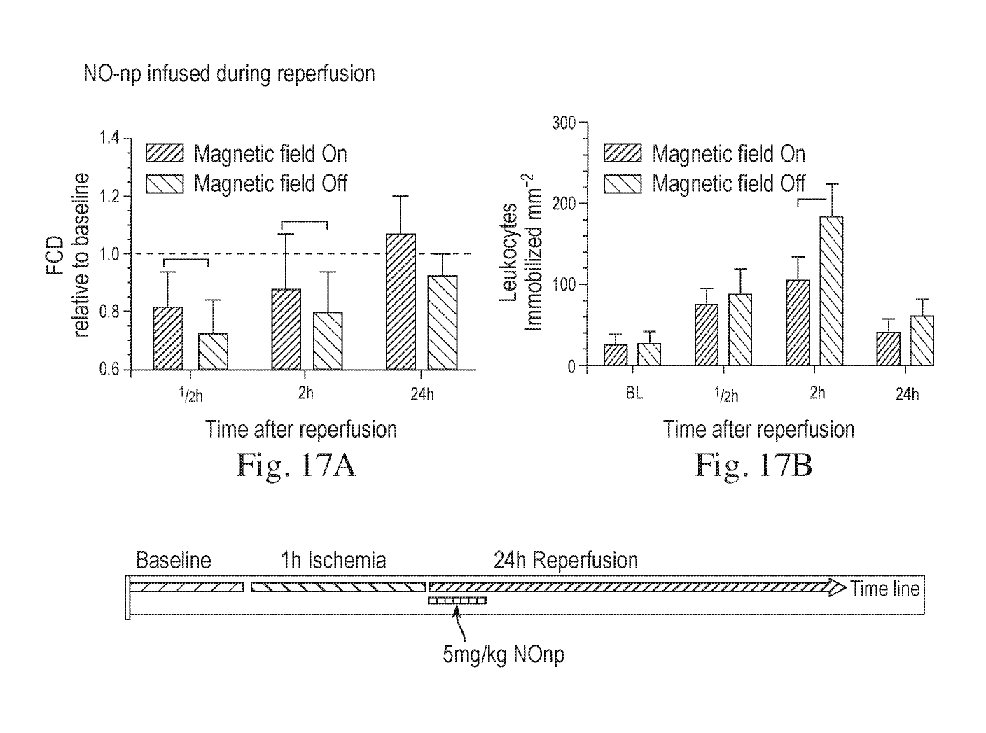

FIGS. 18(A-B). The figure shows that placement of the external magnetic field enhances the ability of infused NO releasing PMNPs to reduce necrosis and apoptosis in the region of IR injury. On and off in the figure refer to with and without the external magnetic field respectively.

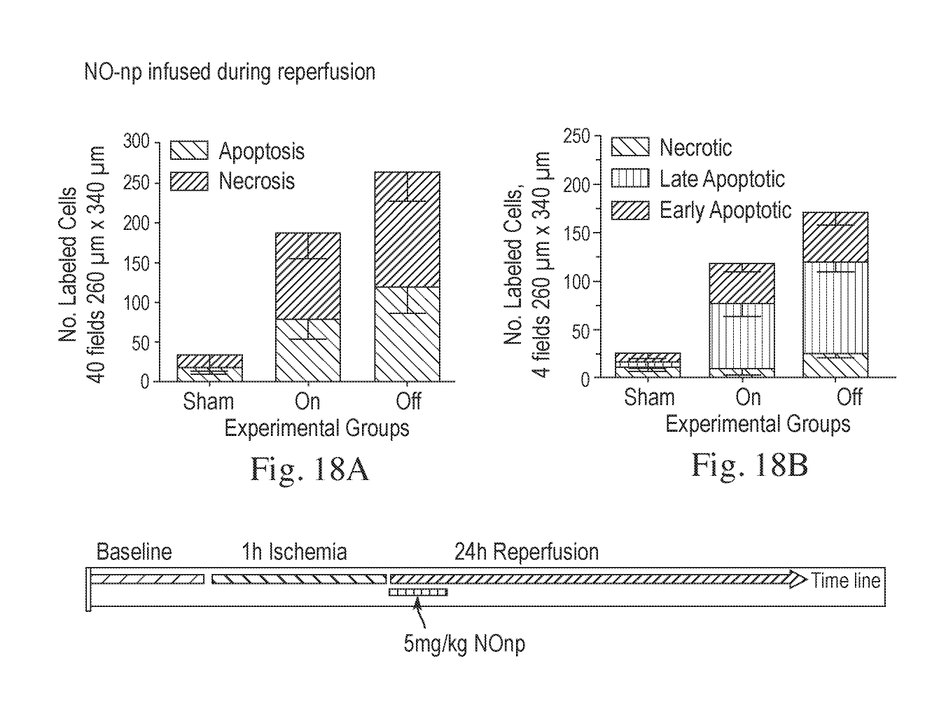

FIG. 19. PMNPs can be used to enhance oxygen tension in magnetic field targeted tissues. The FIG. shows histograms reflecting the measured oxygen levels in specific tissues. The animals are infused with PMNPs loaded with a potent allosteric effector (L35) of hemoglobin that can cross the membrane of red blood cells and cause hemoglobin to unload oxygen. The FIG. shows that for normal tissues the application of the external magnetic field results in enhanced oxygen content of the targeted tissue. In the absence of the magnetic field, there is a decrease in oxygen content due to systemic release of the L35 resulting in a reduced oxygen content of the RBC's at any given site. A detailed account of these results can be found in (Celine Liong et al 2014 Nanotechnology 25 265102 doi:10.1088/0957-4484/25/26/265102). The effect is much greater in tumors and other pathological tissues that manifest leaky blood vessels which will further enhance localization of the PMNPs compared to what occurs in healthy tissues.

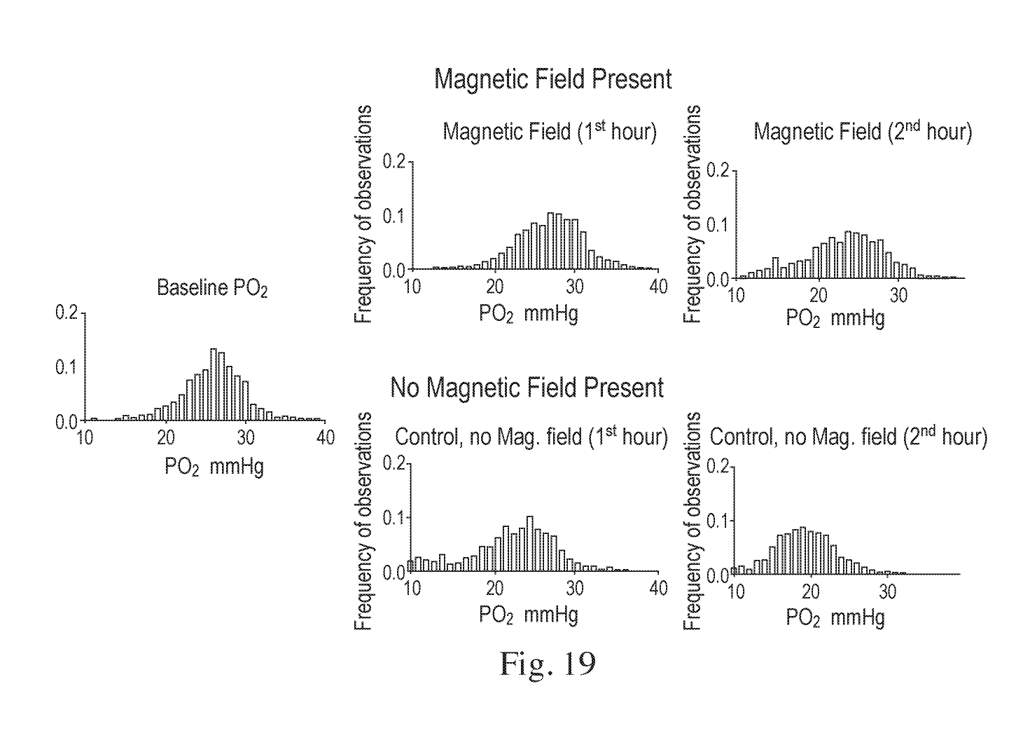

FIG. 20. This figure illustrates the relative efficacy of different drug loaded nanoparticles with respect to cytotoxicity towards U87 glioblastoma cells. This approach allows for facile comparison of drug efficacy either within the same PMNP delivery platform for different drugs or for a given drug in different PMNP platforms. In all cases in it has been shown (using fluorescence labeling) that the PMNPs are rapidly taken up by a wide variety of tumor cells.

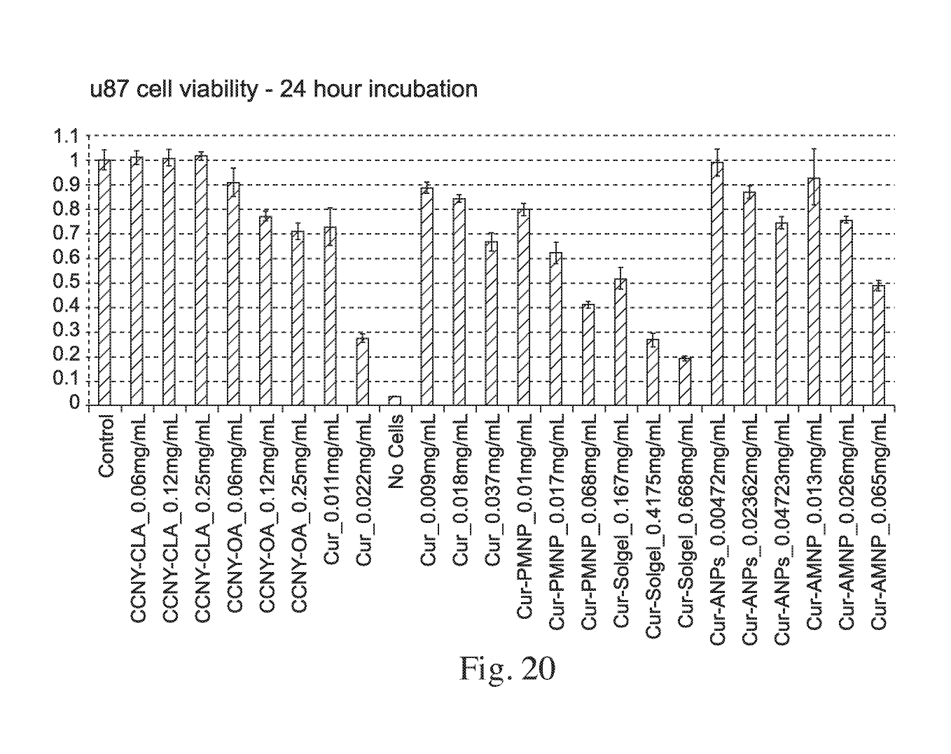

FIG. 21. Fluorescence intensity from adriamycin using two different excitations wavelengths for control solution and supernatant solution after adriamycin treated OA-PMNPs are spun down. The difference between the control and the supernatant provides a direct measure of how much of the added adriamycin has been bound to the OA-PMNP.

FIG. 22 Cytotoxicity of naked curcumin, curcumin conjugated PMNPs and Sol-Gel Nps and blanks (without curcumin) performed on U87 human glioblastoma cells after 48 hours of incubation. Note that 1 μM equivalent curcumin concentration corresponds to 16.7 μg/mL of nanoparticles. The asterisk (*) denotes significant difference with respect to control cells for curcumin and curmumin and NPs conjugated to curcumin and the pound sign (#) denotes significant difference with respect to control cells for the blank NPs. The data represents the mean±SE, n=3 to 18.

### 5. DETAILED DESCRIPTION OF THE INVENTION

### 5.1 Method of Making the PMNP

A platform has been developed for the rapid preparation of paramagnetic nanoparticles (PMNPs) (e.g. comprising iron oxide or gadolinium oxide) of varying sizes from a few nanometers up to micron diameters. In certain embodiment, the coating comprises fatty acids including oleic acid, conjugated linoleic acid and nitro-fatty acids. The method of coating allows for a substantial lipid layer of controllable thickness on the surface of paramagnetic nanoparticles. The thickness of the fatty acid coating is sufficient to allow for the rapid and substantial loading of therapeutically effective amounts of therapeutic drugs. In some embodiments, the therapeutic drugs are lipophilic entities such as lipophilic molecules including many chemotherapeutics and other drugs as well as plasmids.

The coating strategy provided herein can be combined with the incorporation of polyethylene glycol (PEG) molecules onto the surface through the use of PEG chains derivatized on one end with a negatively charged group such as carboxyls. The phospholid derivative PEG, PEG-DSPE is especially effective with respect to being incorporated into the fatty acid coating of the PMNPs. The platform allows for the facile incorporation of the PEG at the time of usage. The use of PEG chains that are also derivatized at the other end of the PEG chain (vis a vis the negatively charged end) allows for the additional attachment of targeting molecules and imaging agents. The resulting coated and loaded PMNPs (loaded with the lipophilic chemotherapeutics, drugs etc.) are effective both therapeutically and diagnostically, such as MRI contrast agents and as drug delivery vehicles. The coated PMNPs can be directed to specific sites using an external magnetic field resulting in highly effective targeted imaging or targeted drug delivery, or both. Magnetic field effects create sustained macro-localization, but the addition of tissue/cell specific targeting molecules allow for further micro-targeting within the macro-domain defined by the magnetic field. Effective (tumor shrinkage without systemic side effects) targeted drug delivery under a magnetic field is described herein in mouse models for breast cancers (using PEGylated Adriamycin loaded OA-coated GdO PMNPs), prostate cancers (using PEGylated Adriamycin loaded OA-coated GdO PMNPs), and glioblastoma (using PEGylated curcumin loaded OA-coated GdO PMNPs). Previous strategies of coating PMNPs with oleic acid result in very small nanoparticles (<20 nm) that have limited drug carrying capacity. The present method uses a new coating platform on much larger nanoparticles (˜100-200 nm) indicate that the enhanced drug carrying capacity in addition to being able to rapidly localize at the target site (within minutes) subsequent to IV infusion creates a new modality for drug delivery of high very effective drug doses to targeted tissues without systemic effects (demonstrated for a three week regimen (infusions/magnet field exposure every third day) Adriamycin loaded OA-PMNPs that resulted in tumor death but no sign of any organ damage (5 animals)). In certain embodiment, the concentration of Adriamycin on PMNPs is 5-10 μg, 10-15 μg, 15-20 μg, 20-25 μg, 25-30 μg, 30-35 μg, 35-40 μg, 40-45 μg, 45-50 μg, 50-55 μg, 55-60 μg, 60-65 μg, 65-70 μg, 70-75 μg, 75-80 μg, 80-85 μg, 85-90 μg, 90-100 μg, or 25-40 μg/mg of OA-PMNP.

In one embodiment, the modified PMNP are coated with oleic acid. This approach is especially useful for loading lipophilic drugs onto magnetic nanoparticles since the oleic acid coating can be loaded with a lipophilic drug. The oleic acid coating-based platform appears far more flexible and versatile than any other platform the inventors are aware of. In embodiments, the PMNPs are finished nanoparticles coated with oleic acid or other fatty acids which are then further loaded with a chemotherapeutic, a drug, an allosteric effector of hemoglobin, plasmid, siRNA or PEG chains that may contain a cell-surface targeting molecule or imaging agent. In an embodiment, the PMNP are coated with more than one of the aforementioned entities. In certain embodiments, the concentration of the plasmid is 5-10 μg, 10-15 μg, 15-20 μg, 20-25 μg, 25-30 μg, 30-35 μg, 35-40 μg, 40-45 μg, 45-50 μg, 50-55 μg, 55-60 μg, 60-65 μg, 65-70 μg, 70-75 μg, 75-80 μg, 80-85 μg, 85-90 μg, 90-100 μg, or 25-40 μg/mg of OA-PMNP.

This platform is used for, inter alia, i) enhanced imaging of magnetic field targeted tumors; ii) targeted drug delivery to tumors or other diseased tissues; iii) enhanced drug delivery for drugs with poor solubility; iv) targeted delivery of drug combinations; v) inhibition or reversal of drug resistance by tumors; vi) targeted delivery of anti-inflammatories to difficult to access tissues (e.g. arthritic joints); vi) combined imaging and drug delivery (theranostic); vii) enhanced targeting using the combination of macro (via magnetic field localization and or leaky vessels in tumors) and micro (using tissue/cell specific targeting molecules) targeting strategies and viii) site-specific radioprotection to limit damage to tissue surrounding irradiated tumors.

In one embodiment, provided herein is a method of making modified PMNP comprising the steps of: (i) adding fatty acid to PMNP core to form a mixture; (ii) Sonicating the mixture; (iii) Spinning the sonicated mixture and washing in deionized water; (iv) Drying and lyophilizing the washed mixture to form a powder; (v) mixing the lyophilized powder with a non-aqueous concentrated solution of a therapeutic agent to form a mixture; (vi) sonicating the mixture from step (v); (vii) spinning the sonicated mixture and washing in deionized water.

In certain embodiments, the PMNP core has a size range from 1 to 1000 nm, 1 to 500 nm, 1 to 100 nm, 1 to 20 nm and 4 to 5 nm, with a standard deviation of 1-4%, 5-10%, 10-20%, 20-30%, and 30-40%. Even very fine and uniform dispersions of the nanoparticles in other carriers or materials are possible.

In one embodiment, provided herein is a method of making a modified PMNP comprising the steps of: (i) adding fatty acid to PMNP core to form a mixture; (ii) Sonicating the mixture; (iii) Spinning the sonicated mixture and washing in deionized water to form an aqueous suspension; (iv) mixing the aqueous suspension with a non-aqueous concentrated solution comprising a therapeutic agent; (v) sonicating the mixture from step (vi); (vi) removing the non-aqueous solvent. In certain embodiments, the therapeutic agent is Adriamycin, taxol, curcumin, dasationib, melanin, allosteric effector, albumin, plasmid, siRNA or a combination thereof.

In certain embodiment, the method further comprises adding methoxy PEG-DSPE, fluorescence-labeled PEG-DSPE, or a derivatized PEG-DSP to the sonicated mixture. In certain embodiments, the PEG-DSPE comprises a reactive species including maleimide, amine, thiol or a combination thereof. In certain embodiment, the reactive species is attached to a fluorophore, PET imaging agent, peptide, antibody, aptamer, contrasting agent or a combination thereof. In certain embodiment, the peptide is CXCR4 antagonistic peptide.

In one embodiment, provided herein is a method of making a drug-loaded albumin-coated paramagnetic nanoparticle (alb-PMNP) comprising the steps of: (i) mixing an ethanol in methanol solution comprising PMNP core with a methanol solution comprising a therapeutic agent to form a mixture; (ii) sonicating the mixture; (iii) adding an aqueous solution comprising albumin to the sonicated mixture. In certain embodiments, the method further comprises the step of: (i) adding methoxy PEG-DSPE, fluorescence-labeled PEG-DSPE, or a derivatized PEG-DSP to the sonicated mixture in step (iii). In certain embodiments, the PEG-DSPE comprises a reactive species including maleimide, amine, thiol or a combination thereof. In certain embodiments, the reactive species is attached to a detectable agent such as a fluorophore, PET imaging agent, peptide, antibody, aptamer, contrasting agent or a combination thereof. In certain embodiments, the peptide is CXCR4 antagonistic peptide.

In one embodiment, provided herein is a method of making modified PMNP comprising the steps of: (i) adding 3-mercaptopropyl-trimethoxysilane (3MPTS) or (N-(2-Aminoethoxyl)-11-Aminoundec-yl trimethoxysilane) (APTS) to a solution containing PMNP core in deionized water to form a mixture; (ii) Sonicating the mixture in step (i); (iv) incubating the sonicated mixture at 4° C.; (v) washing the mixture in deionized water; and (vi) adding 4′-dithiodipyridine (4-PDS) to form a mixture. In certain embodiments, the method further comprises the step of adding 2-imminothiolane & mal-PEG-5K to the mixture of step (vi). In certain embodiment, the method further comprises the step of: (i) adding dithiothreitol (DTT) to the mixture of step (vi); (ii) treating the mixture with buffer saturated with pure NO gas. In certain embodiment, the PMNP core comprises substantially of Gd₂O₃ or iron oxide. In certain embodiments, the Gd₂O₃ is doped with europium or other lanthanides. In certain embodiments, the fatty acid is oleic acid.

In one embodiment, provided herein is a method of making a drug-loaded albumin-coated paramagnetic nanoparticle composition comprising admixing and sonicating (a) a solution of paramagnetic nanoparticles in an ethanol in methanol solution with (b) a solution of the drug to be loaded in methanol, and adding an albumin in aqueous solution to the admixture of (a) and (b) so as to make the drug-loaded albumin-coated paramagnetic nanoparticle composition.

In an embodiment, the process further comprises recovering the drug-loaded albumin-coated paramagnetic nanoparticles comprising applying a magnetic field to separate the drug-loaded albumin-coated paramagnetic nanoparticles from other components of the admixture. In an embodiment of the process, the drug is abraxane or taxol, doxorubicin, circumin, or dasatinib.

### 5.2 Modified PMNP

The invention provides a paramagnetic nanoparticle core comprising a coating with oleic acid or fatty acids (e.g. conjugated linoleic acids including nitro-fatty acids) or a combination thereof. In certain embodiment, the fatty acids are C4-C10, C10-15, C15-C20, C20-C25, C25-C30, C30-C35, C35-C40 fatty acids or combination thereof. In certain embodiments, the fatty acids are not C4-C25. In certain embodiments, the fatty acids are C4-C25. In certain embodiments, the modified PMNP comprises 10-20 μg, 20-25 μg, 25-30 μg, 30-35 μg, 35-40 μg, 40-45 μg, 50-100 μg of therapeutic agent per mg of PMNPs. The coating further comprises lipophilic drugs including chemotherapeutics (e.g. Adriamycin), curcumin, curcumin derivatives, melanin, pro-inflammatory molecules, anti-inflammatory drugs or a combination thereof. In certain embodiments, the coating further comprises plasmids, siRNA, nucleic acid molecules or a combination thereof. In certain embodiments, the coating further comprises lipophilic allosteric effectors of hemoglobin (e.g. L35), or a combination thereof. In certain embodiments, the coating comprises PEG or derivatized PEG of varying size attached to the surface of the nanoparticle core. In certain embodiments, the PEG or derivatized PEG are linked to imaging agents, peptides, aptamers, proteins, water soluble therapeutics, tissue specific targeting agents or a combination thereof. In certain embodiments, the paramagnetic nanoparticle core comprising a coating which comprises albumin and a pharmaceutical or chemotherapeutic agent attached thereto.

In certain embodiments, the modified PMNP comprises 10-20, 20-30, 30-40, 40-50, 50-60, 60-70, 70-80, 80-90, 90-100 μg of therapeutic agent per mg of PMNP. In certain embodiments, the modified PMNP comprises 22-44, 24-40, 50-60 μg of therapeutic agent per mg of PMNP.

In certain embodiments, the modified PMNP comprises 10-20, 20-30, 30-40, 40-50, 50-60, 60-70, 70-80, 80-90, 90-100 μg of therapeutic agent per mg of PMNP per unit time. In certain embodiments, the modified PMNP comprises 22-44, 24-40, 50-60 μg of therapeutic agent per mg of PMNP per unit time. In certain embodiment, the unit time is 1-5, 5-10, 10-15, 15-20, 20-25, 25-30, 30-35, 35-40, 40-45, 45-50, 50-60 secs, 1-2 mins, 2-5 mins, 5-10 mins, 10-30 mins, 30-60 mins.

In certain embodiments, the modified PMNP have a core size of 50-60, 60-70, 70-80, 80-90, 90-100, 100-110, 110-120, 120-130, 130-140, 140-150, 150-160, 160-170, 170-180, 180-190, 190-200, 200-300, 300-400, and 400-500 nm. In certain embodiment, the modified PMNP have a core size of 70-150 nm.

In certain embodiments, the modified PMNP comprises 2, 3, 4, 5, 6, 7, 8, 9, 10, 20, 30, 40, 50 folds more therapeutic agents than PMNP that does not have the modification described in the present disclosure.

In certain embodiments, the PMNP as disclosed herein have improved permeability crossing the blood brain barrier as compared to other PMNP having similar size. In certain embodiments, the PMNP have a nanoparticle core that has similar size as other previously known PMNP and yet has an increased permeability crossing the blood brain barrier by the order of at least 10, 10-10², 10²-10³, 10³-10⁴, 10⁴-10⁵. In certain embodiments, the modified PMNP are 2, 3, 4, 5, 6, 7, 8, 9, 10, 20, 30, 40, 50 folds more efficient in penetration across the blood brain barrier than PMNP that does not have the modification described in the present disclosure. This property is a surprising finding which is imparted by the coating on the nanoparticle core and the amphiphilic polymer on the surface of the coating. Disclosure of the modified PMNP with these properties has not been reported previously.

In certain embodiments, the modified PMNP are 2, 3, 4, 5, 6, 7, 8, 9, 10, 20, 30, 40, 50 folds more efficient in entering a cell at the location that the modified PMNP are targeted in a subject than PMNP that does not have the modification described in the present disclosure. In certain embodiments, the cells are cancer cells. In certain embodiments, the cells are glioblastoma cells. In certain embodiments, the cells are cardiac cells, blood vessel cells and capillary cells. In certain embodiments, the cells are bone marrow, spleen, brain, bone.

In certain embodiments, the modified PMNP have a size dispersion of 0-5%, 5-15%, 15-20%, 20-25% and 25-30%. In certain embodiments, the modified PMNP have a size dispersion of less than 1%. In certain embodiments, the modified PMNP have a size dispersion of less than 0.1%.

A platform has been developed based on PEGylated oleic acid coated gadolinium oxide-based paramagnetic nanoparticles (PMNPs) loaded with potent allosteric effectors of hemoglobin (Hb) which, when released from the PMNPs, penetrate the red blood cell and significantly lower the oxygen affinity of Hb thereby causing a release of oxygen. The PMNPs can be targeted to specific sites using an external magnetic field to localize the infused PMNPs. The large size of the PMNPs limit loss of the circulating PMNPs due to scavenging and extravasation. In certain embodiments, the PMNPs is 10-20 nm, 20-30 nm, 30-40 nm, 40-50 nm, 50-60 nm, 60-70 nm, 70-80 nm, 80-90 nm, 90-100 nm, 100-200 nm, 200-300 nm, or 300-400 nm. The placement of the external magnetic field causes extravastion and resulting localization even in normal tissues. The effect is much greater in tissues manifesting leaky vasculature as in many tumors. The localized PMNP then slowly release the allosteric effector causing an increase in oxygen release in that magnetic field targeted region. This is especially useful as many diseased or damaged tissues are excessively hypoxic and should obviate the need for expensive and often hard to access hyperbaric chamber therapy. Low oxygen concentration in certain tumors limits the efficacy of radiation therapy. The present disclosure provides means to selectively enhance oxygen levels in targeted tissues. In the case of tumors, enhanced oxygenation is understood to increase the efficacy of radiation therapy and some chemotherapies. The present disclosure is also useful for treatment of sickle cell crisis and transplant organ preservation.

In certain embodiments, the modified PMNP comprise hemoglobin allosteric effector which are administered to a subject in need thereof to increase tissue blood oxygen affinity in a tissue or in a cell. In certain embodiments, the modified PMNP increase the tissue blood oxygen affinity by 2, 3, 4, 5, 6, 7, 8, 9, 10, 20, 30, 40, 50 folds at the location that the modified PMNP are targeted in a subject than untreated subject. In certain embodiments, the subject is subjected to a magnetic field. In certain embodiments, the subject is not subjected to a magnetic field. PMNP that does not have the modification described in the present disclosure.

In an embodiment, the paramagnetic nanoparticle(s) are comprised substantially of Gd₂O₃. In an embodiment, the paramagnetic nanoparticle(s) are comprised substantially of Gd₂O₃ nanocrystals. In an embodiment, the Gd₂O₃ is doped with europium. In an embodiment, the Gd₂O₃ is doped with Tb³⁺. In an embodiment, the paramagnetic nanoparticle(s) are comprised substantially of an iron oxide. In an embodiment, the paramagnetic nanoparticle(s) are comprised substantially of magnetite or maghemite.

In an embodiment, the small organic molecule is a pharmaceutical. In an embodiment, the small organic molecule is a chemotherapeutic. In an embodiment, the small organic molecule is lipophilic. In an embodiment, the chemotherapeutic is abraxane, taxol, doxorubicin, curcumin, or dasatinib.

In an embodiment, the external coating comprises a polymer which is a polyethylene glycol (PEG). In an embodiment, the polymer is 1,2-distearoyl-sn-glycero-3-phosphoethanolamine-N-[amino(polyethylene glycol)-2000] (PEG-DSPE). In an embodiment, the polymer has attached to an end thereof, which end is not an end directly attached to the nanoparticle, a fluorophore, PET imaging agent, MRI contrast agent, peptide, a bisphosphonate, antibody, an antigen-binding fragment of an antibody or an aptamer, a pharmaceutical or a chemotherapeutic.

In an embodiment, the external coating comprises a small organic molecule which is oleic acid or other fatty acids such linoleic acid, conjugated linoleic acid, nitro-fatty acids and combinations thereof.

In an embodiment, the external coating of the nanoparticle comprises oleic acid or other suitable fatty acids including nitro-fatty acids and combinations thereof. In an embodiment, the oleic acid substantially coats the external surface of the nanoparticle. In an embodiment, the oleic acid coating also comprises mixed therein a pharmaceutical or a chemotherapeutic. In an embodiment, the pharmaceutical is a lipophilic drug. In an embodiment, the pharmaceutical is a small organic molecule.

In an embodiment, the allosteric effector of hemoglobin is affixed to an external surface of the nanoparticle. In an embodiment, the allosteric effector comprises [3,4,5-trichlorophenylureido-phenoxy]-methylpropionic acid.

In an embodiment, the external coating comprises a blood protein that is albumin. In an embodiment, the albumin has a small organic molecule bound thereto. In an embodiment, the small organic molecule is a pharmaceutical. In an embodiment, the small organic molecule is a lipophilic drug. In an embodiment, the small organic molecule is a chemotherapeutic. In an embodiment, the small organic molecule is paclitaxel.

### 5.2.1 OA-PMNP

Paramagnetic nanoparticles such as those derived from iron oxide or gadolinium oxide are much more effective starting point for targeted drug delivery than liposome-based nanoparticles. Paramagnetic particles are not magnets but are attracted to a magnetic field. The oleic acid-coated paramagnetic nanoparticle (OA-PMNP) based approach disclosed here has features that provide several advantages over the liposome strategy. These include inexpensive method for coating of different types of PMNPs, ease of production, highly flexible platform with respect to both tuning surface properties of the PMNPs and subsequent loading of therapeutics, intrinsically suitable for MRI and CAT scan imaging and can easily be modified to accommodate micro-PET and fluorescence based imaging, can easily control the thickness of the coating, post-preparative modifications to add additional therapeutic molecules, targeting molecules (peptides, aptamers) and circulation enhancement molecules (PEG), intrinsic capacity for targeted delivery based on magnetic field induced localization, ideal for targeted theranostics (combination of targeted drug delivery and imaging of targeted site before, during and after drug delivery).

Combining macro and micro targeting of tissues for both drug delivery and imaging (theranostics) is very effective. Macro-localization at a given site is primarily achieved using magnetic field induced localization of infused PMNPs. PMNPs with suitable coatings will spontaneously localize in the leaky defective vasculature of many tumors thus providing an additional or complementary pathway for macro-localization. Micro-localization within the macro-targeted domain is achieved using cell-specific targeting molecules or tissue-specific targeting molecules attached to the surface of OA-PMNPs. This enhances the drug transport/delivery to targeted tissues with minimal systemic side effects.

The oleic acid coatings are suitable for both iron oxide-based nanoparticles (often referred to as superparamagnetic iron oxide nanoparticles or SPRIONS); however, gadolinium oxide paramagnetic nanoparticles have certain advantages over SPRIONs as well as other highly desirable features. The positive features associated with gadolium oxide based PMNPs include: highly effective MRI contrast agent suitable for existing and widely used positive contrast T1 relaxation based MRI technology which is the standard approach for currently available clinical MRI instrumentation. SPRIONS provide T2 relaxation based negative contrast imaging. Paramagnetic properties can be tuned based on preparative platform (e.g. doping with other lanthanide ions such Eu³⁺ and Tb³⁺ that can enhance both paramagnetic and luminescence properties of the PMNP, and this is within the scope of the present invention). Size can be easily manipulated which in turn influences the paramagnetism (the attraction to a magnetic field). The size of SPRIONS are not easily manipulated. PMNP readily and tightly binds molecules containing carboxylic acid groups and other negatively charged groups. PMNP can easily be coated with different organo-silanes allowing for the introduction of different charges and different reactive groups on the surface (e.g amino, carboxyl, thiols, amines and aldehydes). The gadolinium oxide-based particles do not have the toxicity issues associated with current gadolinium ion (Gd³⁺)-based MRI contrast agents that are based on chelated forms of Gd³⁺. The gadolinium in current chelate-based contrast agents such as MAGNEVIST can get released from the chelate and cause tissue damage (the kidney in particular). The gadolinium in the PMNP is covalently bound up in a crystal lattice as an oxide and as such cannot be released as a free toxic gadolinium ion under physiological conditions as in the chelated products.

Oleic acid-coated PMNPs (OA-PMNPs): Oleic acid has a carboxylic acid group that will strongly bind to the surface of the PMNPs. An oleic acid coating will allow for the facile insertion of lipophilic molecules or lipophilic side chains of large complex molecules into the surface coating on the PMNP. Drug-loaded OA-PMNPs: Adriamycin (doxorubicin) is a potent chemotherapeutic drug but with substantial systemic toxicity that limits the amount that can be given to patient at any given time. As with many other chemotherapeutics, intrinsic or progressive drug resistance by tumors also poses a limitation with respect therapeutic efficacy for adriamycin. Curcumin is emerging as a potential wonder drug with no discernible toxicity even at very high levels. It has potent antioxidant and anti-inflammatory properties. Most significantly it has a broad spectrum of anti-cancer properties that includes the killing of primary and metastatic tumors, inhibition of metastasis, interacting synergistically with other chemotherapeutics and inhibiting/reversing drug resistance to tumors (facilitating the transition from the drug induced tumor senescence state into the therapeutically more effective apoptotic state). The use of curcumin as a therapeutic is limited by the very poor bio-availability of administered curcumin in part due to its very low solubility in water. Herein it is founde have found that curcumin can be bound directly to the surface of the gadolinium oxide PMNP but that binding blocks the surface thus precluding other additions. In addition the resulting PMNPs are prone to aggregation. It also appears that the direct interaction of the curcumin with the surface modifies the chemical and physical properties of the curcumin possible through a redox reaction. Combination of adriamycin and curcuma: Curcumin enhances adriamycin efficacy as a chemotherapeutic. Taxol and taxol-related drugs: These potent lipophilic drugs have substantial issues with respect to toxic systemic side effects that limit dosage. Additionally, intrinsic and progressive drug resistance is also a common issue with these drugs. Targeted delivery and combined delivery with curcumin can greatly increase the efficacy of this family of drugs by overcoming the above limitations. Melanin: results herein using melanin-loaded nanoparticles showed that when introduced and localized subcutaneously, they can provide significant protection from levels of radiation typically used in the treatment of malignancies. Melanin-loaded OA-PMNPs would allow for infusion of the melanin and achieving localization through a directed magnetic field.

Further modifications of OA-PMNPs: PEGylation—the attachment of PEG (polyethylene glycol) to the OA-MNP will improve circulation time and enhance targeting of the “leaky” blood vessels associated with many tumors. Attaching an activated PEG (PEG with a reactive molecular species (e.g. amine, amino, maleimide, sulfhydryl) on the exposed end will also allow for easy attachment of tissue targeting molecules to the PMNP bound PEG. The choice of size for the PEG molecule allows for the attached targeting molecule to extend variable distances beyond the oleic acid coating of the PMNP. PEG can be attached to the OA-PMNP by using a PEG attached either to a phospholipid (PEG-phospholipid) or oleic acid (PEG-OA). The phospholipid moiety enters the oleic acid layer and anchors the extended PEG chain to the surface of the PMNP. The PEG-OA attaches to the surface of the PMNP in the same manner as free OA. The exposed end of the PEG chain containing the reactive molecular species can be used to attach water soluble targeting molecules such as peptides, antibodies and aptamers) but still allows the incorporation of lipophilic chemotherapeutics (e.g. adriamycin, curcumin, taxol) into the oleic acid layer.

Targeting molecules: aptamers are small nucleic acid chains that can be designed to target different cell types (e.g. by SELEX). The aptamers can be synthesized with a reactive thiol that can be readily attached to a maleimide activated PEG chain. Peptides: there are many known targeting peptides that facilitate uptake to different types of cell. Peptides can be easily attached to either amino or maleimide activated PEG chains. Bisphosphonates: these molecules target bone lesions in the area of metastatic bone disease. Antibodies and their antigen-binding fragments, including ScFv—these are well-established ways of targeting a cell or tissue exhibiting a specific cell-surface target molecule to which the antibody can be directed.

### 5.3 Composition Comprising PMNP

The present compositions disclosed herein contain a therapeutically effective amount of a modified PMNP, optionally more than one modified PMNP, preferably in purified form, together with a suitable amount of a pharmaceutically acceptable vehicle so as to provide the form for proper administration to the patient. In certain embodiments, the modified PMNP are administered to a subject using a therapeutically effective regiment or protocol. In certain embodiments, the modified PMNP are also prophylactic agents. In certain embodiments, the modified PMNP are administered to a subject or patient using a prophylactically effective regimen or protocol.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. In certain embodiments, an elderly human, human adult, human child, human infant. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the modified PMNP and pharmaceutically acceptable vehicles are preferably sterile. Water is a preferred vehicle when the modified PMNP is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present composition comprising the modified PMNP, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical vehicles are described in “Remington's Pharmaceutical Sciences” by E. W. Martin.

In a preferred embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the modified PMNP is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

Also provided is a composition comprising a paramagnetic nanoparticle having an external coating comprising an albumin having a pharmaceutical or chemotherapeutic attached thereto, or comprises a paramagnetic nanoparticle having an external coating comprising an oleic acid having a lipophilic pharmaceutical mixed therewith or comprising an oleic acid having a chemotherapeutic mixed therewith.

In an embodiment, the composition comprises a plurality of the paramagnetic nanoparticle. In certain embodiments, the composition contains 1-5%, 5-10%, 10-20%, 20-30%, 30-40% modified PMNP.

### 5.4 Method of Delivering the Modified PMNP

Also provided is a method of delivering a small organic molecule, a polymer, a blood protein, nitric oxide or nitric oxide-releasing compound, oleic acid, an oleic acid having admixed therewith a lipophilic pharmaceutical, or an allosteric effector of hemoglobin, to a predetermined location in a subject comprising administering to the subject a composition as described herein, or a composition comprising nitric oxide-releasing nanoparticles or nitric oxide-releasing compound-releasing nanoparticles, and applying a magnetic field to the subject, such that the magnetic field is present in the predetermined location at a strength sufficient to attract an administered paramagnetic nanoparticle composition.

In an embodiment, the applied magnetic field thereby delivers a small organic molecule, a polymer, a blood protein, nitric oxide or a nitric oxide-releasing compound, oleic acid, a lipophilic pharmaceutical, or an allosteric effector of hemoglobin to the predetermined location.

In an embodiment, the composition is administered systemically. In an embodiment, the composition is administered intravenously, by direct injection or catheterization into the predetermined location or in the vicinity thereof. In an embodiment, the magnetic field is applied from one or more magnetic field external to the body of the subject. In an embodiment, the location of the paramagnetic nanoparticles is monitored using MRI. In an embodiment, the paramagnetic nanoparticles comprise fluorophores.

### 5.5 Therapeutic Uses of the Modified PMNP

A composition comprising the modified PMNP and a pharmaceutically acceptable vehicle, is administered to a patient, preferably a human, to treat ischemic tissue with low tissue perfusion, enhance drug and oxygen delivery to poorly perfused tissues, treat cariogenic shock, prevent ischemia reperfusion injury in targeted tissues, normalize nitric oxide gradient in tumor vasculature and thus reestablish healthy vessels in tumors which limits tumor growth and metastasis while enhancing tumor oxygenation and drug delivery, enhance oxygen content of tumors and thus improve efficacy of radiation and chemotherapy. In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In certain embodiments, the compositions comprising the modified PMNP are administered to a patient, preferably a human, as a preventative measure against such diseases. As used herein, “prevention” or “preventing” refers to a reduction of the risk of acquiring a given disease or disorder. In a preferred mode of the embodiment, the compositions comprising the modified PMNP are administered as a preventative measure to a patient, preferably a human, having a genetic predisposition to the above identified conditions.

In another preferred mode of the embodiment, the compositions comprising the modified PMNP are administered as a preventative measure to a patient having a non-genetic predisposition to the above-identified conditions.

In an embodiment, the method is for treating a cancer and wherein the predetermined location in the subject is a location of the cancer, and wherein the paramagnetic nanoparticles have affixed to an external surface thereof a small organic molecule or a polymer or are coated with an oleic acid having admixed therewith a lipophilic pharmaceutical. In an embodiment, the cancer is a cancer of the breast, nasopharynx, pharynx, lung, bone, brain, sialaden, stomach, esophagus, testes, ovary, uterus, endometrium, liver, small intestine, appendix, colon, rectum, bladder, gall bladder, pancreas, kidney, urinary bladder, breast, cervix, vagina, vulva, prostate, thyroid or skin, head or neck, or is a glioma. In preferred embodiments, the cancer is a pancreatic cancer or central nervous system (CNS) cancer or a cancer in a bone. In an embodiment, the small organic molecule is a chemotherapeutic drug or wherein the polymer has attached thereto a chemotherapeutic drug or the nanoparticles are coated with an oleic acid having admixed therewith a chemotherapeutic drug.

In an embodiment, the small organic molecule is a cytotoxin, which is cytotoxic, or wherein the polymer has attached thereto a cytotoxic drug or the nanoparticles are coated with an oleic acid having admixed therewith a cytotoxic drug. In an embodiment, the cancer is a pancreatic cancer or central nervous system (CNS) cancer or a cancer in a bone. In an embodiment of treating the cancer in the bone, a bisphosphonate as described herein is attached to the paramagnetic nanoparticle. In an embodiment, the bisphosphonate is attached via a PEG attached to an external surface of the paramagnetic nanoparticles. In an embodiment, the bisphosphonate is pamidronate, neridornate, or alendronate.

In an embodiment, the cancer comprises a hypoxic tumor. In an embodiment, the cancer is being treated with another anti-cancer agent and the method enhances the treatment with the other anti-cancer agent. In an embodiment, the anti-cancer agent is radiation therapy. In an embodiment, the anti-cancer agent is a chemotherapy.

In an embodiment, the method is for treating a cancer wherein the predetermined location in the subject is a location of the cancer, and wherein the nanoparticles have affixed to an external surface thereof an allosteric effector of hemoglobin. In an embodiment, the allosteric effector of hemoglobin comprises [3,4,5-trichlorophenylureido-phenoxy]-methylpropionic acid.

Disclosed herein is a method for increasing the oxygen level in a target tissue. Also disclosed is a method of treating a sickle cell disease, and wherein the nanoparticles have affixed to an external surface thereof an allosteric effector of hemoglobin. In an embodiment, the allosteric effector of hemoglobin comprises [3,4,5-trichlorophenylureido-phenoxy]-methylpropionic acid.

In an embodiment of the methods described herein, the nanoparticles further comprise an agent which binds to a cell-surface target. In an embodiment, the agent is an aptamer, an antibody or an antigen-binding fragment of an antibody. In an embodiment, the cell-surface target is present on the cell surface of a cancer cell. In an embodiment, the cell-surface target is preferentially present on a cancer cell over a non-cancerous cell. In an embodiment, the cell-surface target is present on cells of a tissue which is subject to inflammation. In an embodiment, the tissue is a mammalian joint tissue.

Provided herein is a method for treating or reducing a reperfusion injury and the predetermined location in the subject is a location of reperfusion injury or ischemia, and wherein the nanoparticles are nitric-oxide releasing nanoparticles or nitric-oxide releasing compound-releasing nanoparticles.

Provided herein is a method for treating or reducing cardiogenic shock by enhancing in the region of coronary blockage tissue perfusion (targeted NO delivery) and/or enhancing oxygenation (targeted delivery of allosteric effectors of hemoglobin) and/or enhancing revascularization through targeted delivery of agents that enhance angiogenesis including siRNA, plasmids, peptides, and drugs.

In an embodiment, the method is for treating an inflammation and the predetermined location in the subject is a location of the inflammation.

A composition comprising the modified PMNP can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

### 5.5.1 Types of Disease and Disorders

The present disclosure provides methods of treating or preventing or managing a disease or disorder in humans by administering to humans in need of such treatment or prevention a pharmaceutical composition comprising an amount of modified PMNP effective to treat or prevent the disease or disorder. In other embodiments, the disease or disorder is an inflammatory disease or disorder. In certain embodiments, the present invention encompasses treating patients with glioblastoma by administering to those patients the modified PMNP after those patients have gone into remission following another cancer therapy. In certain embodiments, for those patients in remission, an effective amount of the modified PMNP will be an amount effective to prolong or increase the amount of time before recurrence of the cancer.

The present invention encompasses methods for preventing, treating, managing, and/or ameliorating an inflammatory disorder or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease, patients with broncho-pulmonary dysplasia, patients with congenital heart disease, patients with cystic fibrosis, patients with acquired or congenital heart disease, and patients suffering from an infection), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, or ameliorate a viral respiratory infection or one or more symptoms thereof.

In certain embodiments, the invention provides a method of preventing, treating, managing, and/or ameliorating an autoimmune disorder or one or more symptoms thereof, said method comprising administering to a subject in need thereof a dose of an effective amount of one or more pharmaceutical compositions of the invention. In autoimmune disorders, the immune system triggers an immune response and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress, destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin.

Examples of autoimmune disorders that can be prevented, treated, managed, and/or ameliorated by the methods of the invention include, but are not limited to, adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, discoid lupus, essential mixed cryoglobulinemia, fibromyalgia-fibromyositis, glomerulonephritis (e.g., IgA nephrophathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, juvenile arthritis, lichen planus, lupus erythematosus, Meniere's disease, mixed connective tissue disease, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychrondritis, Polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, takayasu arteritis, temporal arteritis/giant cell arteritis, ulcerative colitis, urticaria, uveitis, Uveitis Opthalmia, vasculitides such as dermatitis herpetiformis vasculitis, vitiligo, and Wegener's granulomatosis.

Any type of cancer can be prevented, treated, and/or managed in accordance with the invention. Non-limiting examples of cancers that can be prevented, treated, and/or managed in accordance with the invention include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; dendritic cell cancer, including plasmacytoid dendritic cell cancer, NK blastic lymphoma (also known as cutaneous NK/T-cell lymphoma and agranular (CD4+/CD56+) dermatologic neoplasms); basophilic leukemia; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The prophylactically and/or therapeutically effective regimens are also useful in the treatment, prevention and/or management of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. In some embodiments, cancers associated with aberrations in apoptosis are prevented, treated and/or managed in accordance with the methods of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are prevented, treated and/or managed in accordance with the methods of the invention. In other specific embodiments, a sarcoma, melanoma, or leukemia is prevented, treated and/or managed in accordance with the methods of the invention. In certain embodiments, the subjects have acute myelogenous leukemia (AML). In certain other embodiments, the subjects have myelodysplastic syndrome (MDS). In other embodiments, the subjects have chronic myelomonocytic leukemia (CMML). In other specific embodiments, myelodysplastic syndrome is prevented, treated and/or managed in accordance with the methods of the invention.

### 5.5.2 Cancer Treatment

A major objective in treatment of cancers is to be able to target the tumor with sufficient levels of the appropriate therapeutic without systemic toxicity. The use of targeting molecules attached to either the therapeutic molecules directly or to nanoparticles containing the therapeutic molecule has not proven to be especially effective. A major pathway for localization of either the free therapeutic molecule or the drug delivery vehicle containing the therapeutic molecule is through the EPR effect (EPR=enhanced permeability and retention) resulting from the leaky vasculature associated with many (but not all) tumors. For the EPR effect to work the circulating drug or delivery vehicle must remain in a functional form in circulation for a sufficiently long time to allow for the build of local concentration at the tumor site via the EPR effect. This build up requires circulation times of at least 8 to 24 hours. Thus, over this several hour period, a drug-loaded nanoparticle has to both avoid being cleared and avoid releasing its therapeutic payload (resulting in potential systemic effects and decreased efficacy at the target site). Herein is disclosed an approach and a biocompatible nanoparticle platform that takes advantage of the EPR effect but drastically shortens the accumulation time from hours to minutes. Drug-loaded paramagnetic nanoparticles (PMNP) (e.g. gadolinium oxide-based) are infused intravenously and then localized at the target site using a strategically placed external magnetic field. Based on imaging studies (both MRI and whole body fluorescence), a several minute treatment with the externally placed magnetic field is sufficient to create persistent localization for many hours once the magnetic field is removed. The persistent retention only occurs for those tissues manifesting the EPR effect. This approach when applied to targeting one of many xenographed tumors with adriamycin-loaded PMNPs results in rapid and effective site specific reduction in tumor size without evidence of either systemic toxicity or tumor shrinkage in non-targeted tumors. The ability to easily modify the PMNP platform to accommodate a wide variety of chemotherapeutic and immunogenic molecules as well cell-specific targeting molecules (peptides, antibodies, bisphosphonates, aptamers), makes this very powerful. Also, the induction of leaky vasculature in EPR resistant tumors through targeted treatments with radiation will likely make these resistant tumors accessible to this approach.

Targeted drug delivery using nanoparticles is a major trend in cancer therapy. Targeted delivery can be expected to minimize systemic toxicity and enhance efficacy by being able to deliver much larger doses of chemotherapeutic drugs directly to the site of the tumor. Tumor targeting using nanoparticles coated with targeting molecules is not very effective in vivo in part due to plasma proteins adhering to the nanoparticles and interfering with the range of motions or accessibility of the targeting molecules. Instead the most promising approaches appear based on utilizing the EPR effect (enhanced penetration and perfusion) arising from the leaky vasculature associated with many tumor types. For those tumors without such vessels, radiation induced inflammation can be used to create “leakiness” and thus render such tumors susceptible to the EPR effect. The EPR effect allows for localized accumulation of circulating nanoparticles over a period of many hours during which time the nano's have to remain in circulation and not release their drug payload. This requirement poses a serious challenge for the design of suitable platforms. This laboratory has shown that the use of paramagnetic nanoparticles (PMNPs) allows for very rapid accumulation of the PMNP's at the tumor site targeted using an externally applied magnetic field. Once initially localized using the external magnetic field, the PMNP's remain trapped for what may well be an indefinite period (at least 24 hours) after the magnetic field is removed. Thus the several hour accumulation time is reduced to minutes using the external magnetic field which can then be removed without concern that the PMNP's will continue to circulate. The PMNP's do not appear to permanently (or even transiently) accumulate in tissues that do not have the leaky vasculature (with or without the externally applied magnetic field). In contrast, the PMNP's do appear to accumulate in EPR sensitive tissues even in the absence of the magnetic field but instead of minutes the accumulation time is much longer as anticipated from many studies on the EPR effect using other types of nanoparticles. Albumin-based nanoparticle appear to be a promising strategy that utilizes the EPR effect. Abraxane is a notable example whereby taxol loaded albumin nanoparticles diminish systemic effects and appear to enhance efficacy by preferentially accumulating in the tumor. Building upon all of the above concepts by developing a general platform that allows for the coating of PMNS's with drug loaded albumin thereby adding the following capabilities and advantages: i) very rapid targeting/localization; ii) imaging; iii) enhanced and more efficient drug loading; and iv) greater plasticity with respect to drugs, combination of drugs and physical properties of the nanoparticles.

Albumin forms a very tight shell/coating around a gadolinium oxide core PMNPs that remains intact in aqueous solutions. Several drugs (curcumin, Adriamycin but not taxol) directly bind to the surface of the PMNP's with high avidity. Albumin can coat the drug loaded PMNP's. Albumin is an effective carrier/transporter for many lipophilic drugs hence both the PMNP and the albumin can be used to carry drugs. Taxol loaded albumin (Abraxane) can be used to coat the PMNP's thus allowing for taxol and related drugs to participate in the targeted delivery. PEG can easily be attached to the surface of the PMNP using PEG-DSPE (1,2-distearoyl-sn-glycero-3-phosphoethanolamine-N-[amino(polyethylene glycol)-2000) derivative. The DSPE moiety has a very high electrostatic attraction for the surface of the gadolinium oxide (GdO) nanoparticles. PEG imparts a stealth quality to nanoparticles allowing them to evade scavenging by macrophages. PEG also enhances the EPR effect making capture in leaky vessels more probable. Bifunctional PEG with one end having the DSPE moiety and the other end a reactive species (e.g. maleimide, amine, thiol) can be used to attach to the PMNP's PEG with fluorophores, PET imaging agents, peptides, antibodies, aptamers, and additional MRI contrast agents (the GdO based PMNPs have intrinsic relaxativity properties that can be tuned and used for positive contrast MRI imaging).

In certain embodiments, the method of treating cancer includes: (i) a reduction of cancer cells, (ii) absence of increase of cancer cells; (iii) a decrease in viability of cancer cells; (iv) decrease in growth of cancer cells, in a subject.

In certain embodiments, the subject that is treated with the present method of the disclosure has been diagnosed with the disease and has undergone therapy. In certain embodiments, the subject that is treated with the present method of the disclosure has been diagnosed with cancer and has undergone cancer therapy.

In certain embodiments, the subject is in remission from cancer. In certain embodiments, the subject has relapsed from cancer. In certain embodiments, the subject has failed cancer treatment.

### 5.5.3 PMNP Delivering Curcumin for the Treatment of CNS Tumor

Malignant CNS tumors are associated with high mortality and morbidity; they remain a challenge mainly because we lack therapeutic agents that readily cross the Blood Brain Barrier (BBB) and accumulate at the tumor site in concentrations that are effective for treatment. In certain embodiments, the modified PMNP of the present disclosure are 10, 10², 10³, 10⁴ or 10⁵ times more efficient in crossing the BBB as compared to other PMNP of similar sizes. In-vitro BBB permeability to two nanoparticle (NP) platforms were tested; a hybrid Sol-Gel/glass (sugar derived) platform and a paramagnetic Gd₂O₃ nanocrystal as core platform (PMNP). NP surface modification with low density 2000 PEG conjugate and a net neutral charge increased the flux of NPs across the BBB. An external magnetic field also increased the flux of paramagnetic NPs suggesting that enhanced in-vivo localization could occur via magnetic field at the tumor site. Both NP platforms were conjugated with Curcumin, the active ingredient in the spice turmeric, which holds outstanding anti-cancer, anti-inflammatory and neuro-protective properties. Curcumin lowered the viability of tumor cells and once conjugated to the NPs, a lower dose was needed to reduce the viability of U87 glioblastoma cancer cells. In addition, using a mouse model of Glioblastoma, delivery of PMNPs to the tumor site was enhanced by placing a magnetic field in the vicinity of the tumor.

### 5.6 Mode of Administration

The present compositions, which comprise one or more modified PMNP, are preferably administered by infusion or bolus injection, by absorption through epithelial or mucocutaneous linings (e.g., oral mucosa, rectal and intestinal mucosa, etc.) or orally and may be administered together with another biologically active agent. Administration can be systemic or local. Various delivery systems are known. In certain embodiments, more than one modified PMNP is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the modified PMNP into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site).

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the modified PMNP, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

### 5.7 Dosage

The amount of a modified PMNP that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In specific preferred embodiments of the invention, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 5 milligrams of modified PMNP per kilogram body weight. The dosage amounts described herein refer to total amounts administered; that is, if more than one modified PMNP is administered, the preferred dosages correspond to the total amount of the modified PMNP administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of modified PMNP per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the modified PMNP for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more modified PMNP. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one modified PMNP. In another embodiment, the kit comprises a modified PMNP and a second therapeutic agent.

The modified PMNP are preferably assayed in vitro and in vivo, for the desired therapeutic or prophylactic activity, prior to use in humans. For example, in vitro assays can be used to determine whether administration of a specific modified PMNP or a combination of modified PMNP is preferred for lowering fatty acid synthesis. The modified PMNP may also be demonstrated to be effective and safe using animal model systems.

Other methods will be known to the skilled artisan and are within the scope of the invention.

### 5.8. Combination Therapy

In certain embodiments, the modified PMNP can be used in combination therapy with at least one other therapeutic agent. The modified PMNP and the therapeutic agent can act additively or, more preferably, synergistically. In a preferred embodiment, a composition comprising a modified PMNP is administered concurrently with the administration of another therapeutic agent, which can be part of the same composition as the modified PMNP or a different composition. In another embodiment, a composition comprising a modified PMNP is administered prior or subsequent to administration of another therapeutic agent. As many of the disorders for which the modified PMNP are useful in treating are chronic disorders, in one embodiment combination therapy involves alternating between administering a composition comprising a modified PMNP and a composition comprising another therapeutic agent, e.g., to minimize the toxicity associated with a particular drug. The duration of administration of each drug or therapeutic agent can be, e.g., one month, three months, six months, or a year. In certain embodiments, when a modified PMNP is administered concurrently with another therapeutic agent that potentially produces adverse side effects including but not limited to toxicity, the therapeutic agent can advantageously be administered at a dose that falls below the threshold at which the adverse side is elicited.

The present modified PMNP can be administered together with treatment with irradiation or one or more chemotherapeutic agents. For irridiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2.nd. Ed., J.B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition comprising the modified PMNP further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition comprising the modified PMNP.

Any therapy (e.g., therapeutic or prophylactic agent) which is useful, has been used, or is currently being used for the prevention, treatment, and/or management of a disorder, e.g., cancer, can be used in compositions and methods of the invention. Therapies (e.g., therapeutic or prophylactic agents) include, but are not limited to, peptides, polypeptides, conjugates, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies and surgery. In certain embodiments, a prophylactically and/or therapeutically effective regimen of the invention comprises the administration of a combination of therapies.

Examples of cancer therapies include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; Bcl-2 inhibitors; Bcl-2 family inhibitors, including ABT-737; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

In some embodiments, the therapy(ies) used in combination with the modified PMNP is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunomide). Other examples of immunomodulatory agents can be found, e.g., in U.S. Publ'n No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent.

In some embodiments, the therapy(ies) used in combination with the modified PMNP is an anti-angiogenic agent. Non-limiting examples of anti-angiogenic agents include proteins, polypeptides, peptides, conjugates, antibodies (e.g., human, humanized, chimeric, monoclonal, polyclonal, Fvs, ScFvs, Fab fragments, F(ab)2 fragments, and antigen-binding fragments thereof) such as antibodies that bind to TNF-alpha, nucleic acid molecules (e.g., antisense molecules or triple helices), organic molecules, inorganic molecules, and small molecules that reduce or inhibit angiogenesis. Other examples of anti-angiogenic agents can be found, e.g., in U.S. Publ′n No. 2005/0002934 A1 at paragraphs 277-282, which is incorporated by reference in its entirety. In other embodiments, the therapy(ies) used in accordance with the invention is not an anti-angiogenic agent.

In some embodiments, the therapy(ies) used in combination with the modified PMNP is an inflammatory agent. Non-limiting examples of anti-inflammatory agents include any anti-inflammatory agent, including agents useful in therapies for inflammatory disorders, well-known to one of skill in the art. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), β₂-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., pro staglandins, thromboxanes, and leukotrienes. In other embodiments, the therapy(ies) used in accordance with the invention is not an anti-inflammatory agent.

In certain embodiments, the therapy(ies) used is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, melphalan, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclines include but are not limited to daunorubicin, doxorubicin, epirubicin, idarubicin, and mitoxantrone. Topoisomerase II inhibitors include, but are not limited to, topotecan, irinotecan, etoposide (VP-16), and teniposide. Mitotic inhibitors include, but are not limited to taxanes (paclitaxel, docetaxel), and the vinca alkaloids (vinblastine, vincristine, and vinorelbine).

In some embodiments, modified PMNP is used in combination with radiation therapy comprising the use of x-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

Currently available cancer therapies and their dosages, routes of administration and recommended usage are known in the art and have been described in such literature as the Physician's Desk Reference (60th ed., 2006). In accordance with the present invention, the dosages and frequency of administration of chemotherapeutic agents are described supra.

Ssss

ssss

### 5.7 Imaging Uses of the Modified PMNP

Also provided is a method of imaging a predetermined location in a subject comprising administering to the subject the composition described herein that comprise an imaging agent and applying a magnetic field to the subject, such that the magnetic field is present in the predetermined location at a strength so as to attract the composition to a predetermined location, and collecting an imaging signal from the predetermined location using an imaging device so as to thereby image the predetermined location. In an embodiment, the predetermined location comprises, or is thought to comprise, a tumor. In an embodiment, the predetermined location comprises, or is thought to comprise, inflammation. In an embodiment, the imaging agent is a fluorophore. In an embodiment, the imaging agent is the paramagnetic nanoparticles themselves. In an embodiment, the method treats the tumor by delivering an anti-tumor pharmaceutical or chemotherapeutic to the predetermined location, wherein the paramagnetic nanoparticle composition comprises the anti-tumor pharmaceutical or chemotherapeutic. In an embodiment, the method treats the inflammation by delivering an anti-inflammatory pharmaceutical to the predetermined location, wherein the paramagnetic nanoparticle composition comprises the anti-inflammatory pharmaceutical. In embodiments, the anti-inflammatory pharmaceutical, anti-tumor pharmaceutical or chemotherapeutic are part of the paramagnetic nanoparticle composition via being admixed with an oleic acid coating of the paramagnetic nanoparticles or by being attached to an albumin coating of the paramagnetic nanoparticles.

All combinations of the various elements described herein are within the scope of the invention unless otherwise indicated herein or otherwise clearly contradicted by context.

The invention is illustrated in the following sections, which is set forth to aid in the understanding of the invention, and should not be construed to limit in any way the scope of the invention as defined in the claims that follow thereafter.

### 6. EXAMPLES

### 6.1 Hemoglobin Allosteric Effector Loaded OA-PMNP (L35-PMNP)

The preparation of the hemoglobin allosteric effector (L35) loaded OA-PMNP (L35-PMNP) starts with the preparation of the oleic acid coated paramagnetic nanocrystalline core. 10 mg of the nanocrystalline core (e.g. commercially purchased (Nanostructured & Amorphous Materials, Inc., Houston, Tex. 77084, USA) Gadolinium Oxide (GdO) nanocrystals (with ˜30 nm average diameters). Larger diameter PMNP cores can be used as can other PMNP cores such as doped GdO nanocrystals with enhanced paramagnetism. The PMNP cores are washed several times in 5 ml of deionized (DI) water and then centrifuged. 300 μl of oleic acid in DMSO (1:19) is mixed with water-free spun down particles followed by vigorous sonication for 1 hr. in a cold water bath and then left on lab rotor overnight. Higher or lower concentrations of OA are possible although this concentration provides optimum drug loading properties. The resulting particles are spun down and washed several times with DI water, dried and then lyophilized for storage. They are reconstituted by mixing with an aqueous solvent and sonicate briefly. The resulting suspension is stable with no detectable aggregates forming over an extended several day periods. Preliminary dynamic light scattering measurements indicate that the resulting particles are less that 100 nm. Larger nanoparticles can be prepared by starting with larger PMNP cores. The drug loading process consists of mixing 1 mg/ml of allosteric effector (e.g. L35 or related type molecules) solubilized in 1 ml of DMSO with 10 mg/ml of oleic acid coated gadolinium oxide based paramagnetic nanoparticles (PMNPs) at room temperature. After the DMSO mix is allowed to remain for 24 hrs in the dark at room temperature, the suspension is centrifuged. The brown color of the L-35 is fully localized with the spun down PMNPs. The DMSO is poured off and the brown colored PMNPs washed with PBS buffer and then subjected to vortexing to resuspend the particles. The suspension is again centrifuged, the buffer poured off and the samples vortexed again. The samples are then spun down, excess buffer poured off and the remaining brown material is then lyophilized. The L35 coated PMNPs are then PEGylated by resuspending the lyophilized PMNPs and then adding a small aliquot of m-PEG-DSPE (Nanocs inc. USA) dissolved in DMSO (derived from a stock solution of 1 mg of m-PEG-DSPE (Nanocs inc. USA) dissolved in 1 ml of DMSO). Typically 10-50 μl of the DMSO stock solution is used to conjugate 50-100 mg of L35-PMNPs. Doubly derivatized PEG-DSPE chains can be used to introduce a reactive end to the PEG for attachment of either cell specific targeting molecules (e.g. peptides, aptamers).

TABLE 1
Changes in systemic blood O₂ affinity.

| | | | With | Without |
|---|---|---|---|---|
| | | Untreated | Magnetic Field | Magnetic Field |
| Baseline | P50 (mmHg) | 32.6 ± 1.4 | 32.6 ± 1.4 | 32.6 ± 1.4 |
| | Hill number | 2.94 ± 0.08 | 2.96 ± 0.10 | 2.93 ± 0.12 |
| 1^{st} hour | P50 (mmHg) | 32.6 ± 1.4 | 33.1 ± 1.7 | 38.4 ± 1.4 |
| | Hill number | 2.92 ± 0.10 | 2.84 ± 0.12 | 2.62 ± 0.16 |
| 2^{nd} hour | P50 (mmHg) | 32.6 ± 1.4 | 35.2 ± 1.5 | 39.6 ± 1.0 |
| | Hill number | 2.93 ± 0.11 | 2.72 ± 0.14 | 2.50 ± 0.09 |

L35-PMNPs subjected to a magnetic field increased tissue PO₂s. Without an applied magnetic field, L35-PMNPs decreased tissue PO₂s. The different effects are due to systemic changes in blood O₂ affinity. Target modification of blood O₂ affinity with L35-PMNPs increases PO₂ and limits and delays negative effects of systemic changes in blood O₂ affinity. This technology can enhance cancer therapy to hypoxic tumors or to resolve local hypoxic conditions without disturbing systemic O₂ transport homeostasis.

### 6.2 Platform for Coating Either Iron Oxide or Gadolinium Oxide Nano Crystal Based Paramagnetic Nanoparticles (PMNP) with Oleic Acid (OA-PMNP)

10 mg of PMNPs (either iron oxide based PMNPs or gadolinium oxide based PMNPs with and without other rare earth/lanthanide elements added as dopants, e.g. Eu, Yb and Tb,) are washed several times (which removes excess unreacted materials from the PMNP preparative phase) in 5 ml of deionized (DI) water and then centrifuged. 300 μl of oleic acid (or any other or combination of fatty acids including conjugated fatty acids such as linoleic acid and nitro-fatty acid derivatives of any of the conjugated FA's) in DMSO (1:19) is mixed with water-free spun down particles followed by vigorous sonication for 1 hr. in a cold water bath and then left on lab rotor overnight. The resulting particles were spun down and washed several times with DI water, dried and then lyophilized for storage. They are reconstituted by mixing with an aqueous solvent and sonicating briefly. The resulting suspension is stable with no detectable aggregates forming over an extended a several day period. Dynamic light scattering measurements indicate that the resulting particles have diameters that are in the range 100 nm or less depending on the starting nano-crystal core.

### 6.3 Generalized Strategies for Incorporating Lipophilic/Hydrophobic Molecules into the Oleic Acid (or Other FA) Coating on the Surface of the PMNP

Strategy 1: The powder form of OA-PMNP is mixed with a small volume of a non-aqueous concentrated solution of the to-be-incorporated molecule, and then sonicated, then spun down in a centrifuge followed by washing with DI water.

Strategy 2: Small aliquots of a non-aqueous solution of the to-be-incorporated molecule are slowly added to an aqueous suspension of OA-PMNPs and upon each aliquot addition sonicated followed by procedures suitable to allow for the slow evaporation or removal of the non-aqueous solvent. This process is designed to allow for the transfer of lipophilic/hydrophobic molecules from the non-aqueous solvent to the hydrophobic coating associated with the oleic acid coating of the OA-PMNP without loss of oleic acid to the non-aqueous solvent.

### 6.4 Platform for Drug Loading of OA-PMNP: Adriamycin (Similar for Taxol)

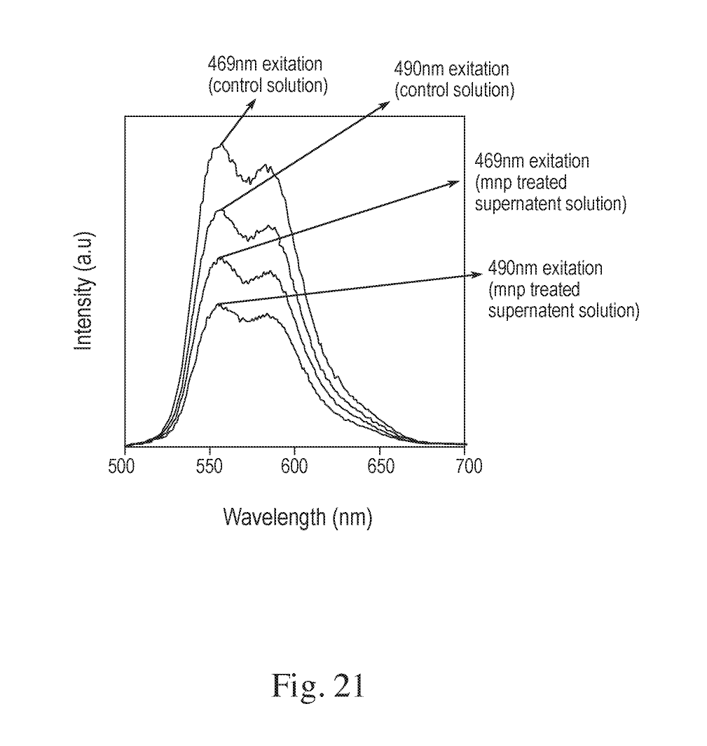

Therapeutic grade adriamycin solution 2 mg/ml is mixed with lyophilized OA-PMNPs, sonicated briefly and left overnight on lab rotor. The resulting coated particles were spun down and washed several times with DI water, dried and then lyophilized for storage. The particles are now brightly colored with the adriamycin. The difference in the intensity of the fluorescence spectra from adriamycin in the starting stock solution and in the supernatant solution after spinning down the adriamycin treated OA-PMNPs is used to determine how much of the added adriamycin becomes conjugated to the OA-PMNPs (see figure below) This methods indicates that for the coated PMNPs there are 25 microgram of adriamycin/mg of PMNPs. Preliminary adriamycin release profiles for adriamycin coated OA-PMNPs indicate that the release is very slow at pH 7.4 or higher pH values but increases dramatically as the pH drops. This pH dependence is ideal for drug delivery to the acidic environment associated with many tumors. The addition of PEG into the coating is readily achieved using the protocol described above for the L35 loaded OA-PMNP. See FIG. 21.

### 6.5 Platform for Drug Loading of OA-PMNP: Curcumin

10 mg of dry OA-PMNPs were treated with 100 μl of 20 mg/ml of curcumin in DMSO and sonicated for 15 minutes. The treated particles were then spun down followed by several washings with DI water. The estimated curcumin content on OA-PMNPs is between 30-44 micrograms/mg of OA-PMNP. Curcumin can be directly attached to the surface of the oleic acid free PMNP but the resulting particles tend to aggregate and are not amenable to further modifications/drug additions. PEG addition is achieved as described above.

### 6.6 Curcumin-Adriamycin Coated OA-PMNPs

10 mg of lyophilized curcumin conjugated OA-PMNPs were treated with the commercial 2 mg/ml adriamycin solution and mixed on lab rotor overnight. The resultant particles were washed with DI water, dried and then lyophilized for storage.

### 6.7 PEG Coated OA-PMNPs

A small aliquot of carbon tetrachloride based solution of a phospholipid coupled PEG (e.g. DSPE-PEG, 1,2-distearoyl-sn-glycero-3-phosphoethanolamine-N-[amino(polyethylene glycol)-x where x refers to the size of the PEG) either with or without derivatization of the phospholipid conjugated PEG chain (e.g. with a reactive group such as maleimide, thiols, amines carboxylates, or fluorescent probes), was added to lyophilized OA-PMNP (gadolinium oxide based), sonicated, kept on a rotating mixer for several hours and then diluted with DI water and spun down. This protocol can be applied to any of the drug loaded OA-PMNP materials. The doubly derivatized PEG once added to the OA-PMNP can be modified with targeting molecules (peptides, aptamers) or imaging agents. Addition of the PEG has been shown to minimize aggregation, enhance circulation time (in vivo animal models) and improve crossing rate and efficiency for the blood brain barrier (BBB) in an in vitro model. Application of an external magnet further enhanced the crossing of the BBB.

### 6.8 Other Molecule Loaded OA-PMNPs: Melanin

The same protocol used for curcumin also works for melanin resulting in melanin loaded OA-PMNPs. These particles have potential use as radioprotectants. The melanin loaded OA-PMNPs would be infused into the patient and then magnetically localized in healthy tissue surrounding the diseased tissue targeted for radiation treatment. Our earlier studies indicate that localized melanin can prevent radiation damage in irradiated rat legs. Additionally melanin loaded PMNP can be used as targeted contrast agent for photo-acoustic based imaging.

### 6.9 Other Molecule Loaded OA-PMNPs: Proteins

Albumin readily attach to OA-PMNPs. Fluorescent labeled albumin was used to confirm that the proteins are bound to the OA-PMNPs.

### 6.10 Plasmid Loaded OA-PMNP

Uptake of PMNPs by U87 cells was confirmed by binding an mCherry plasmid to the nanoparticles. A 10 mg/mL OA-PMNP solution was mixed and bound with/to an mCherry plasmid solution (20 μg/mL). U87 Cells were plated on 35 mm Ibidi imaging dishes and grown for two days with 10% DMEM. The cells were then incubated with 1.5 μL of mCherry plasmid-PMNP solution in 1 mL of Optimem. The final concentration of plasmid was 30 pg/mL, corresponding to a 15 ng/mL PMNP solution. After four hours, control cells (without the mCherry-PMNP solution) and experimental cells were washed with PBS and imaged with a 40× dry objective using the same optical variables. The results indicate close to 100% transfection efficacy. Similar results indicative of close to 100% transfection and very high cell uptake efficacy were also obtained using a different plasmid (same preparative protocol and a different cell line (embryonic kidney cell). Current maximum transfection efficacy from commercial materials as at least two times lower.

### 6.11 Thiol Coated PMNPs, Amine Coated PMNPs and PEG Coating for the Amine-Coated PMNPs

Thiol coating: 10 mg of PMNPs are washed several times and dispersed in 5 ml of DI water. 1000 of 3 MPTS (3-mercaptopropyl-trimethoxysilane) is added to the above solution and sonicated and incubated at 4° C. for 2 days. The coated particles were washed several times with DI water and PBS buffer (10 mM 7.4 pH).

The above washed particles are made reacted with 4′-dithiodipyridine (4-PDS) reagent having control, the difference in the absorption co-eff. of supernatant of control to sup recorded @324 μm and thiols were confirmed.

Result: after 20 times dilution of the sup to control, the estimation of thiol is 58.3 μM/mg in the second attempt 61 μM/mg of PMNP.

Amines coating: 10 mg of PMNP s were washed and dispersed in 5 ml of DI water, to the suspension 50 μl of APTS (N-(2-Aminoethoxyl)-11-Aminoundec-yl trimethoxysilane) is added and sonicated, and incubated as above, the coated particles were washed many times with PBS 10 mM pH 7.4. in the second attempt 20 μl of APTS (N-(2-Aminoethoxyl)-11-Aminoundecyl trimethoxysilane) is added and sonicated, and incubated @ 4° C. for 2 days.

PEG coating: To the above amine coated particles a stock solution of 2-imminothiolane & mal-PEG-5K 50 mg/ml in 10 mM PBS pH 7.4, is added, incubated @ 4° C. for 2 days. The particles were spin down and supernatant is collected. PEG coating is confirmed from FPLC between control and supports the observed high level of coating of PEG on the PMNP using other detection techniques (fluorescence). The imminothiolane reacts with the amines on the surface of the PMNP and the maleimide-derivatized PEG reacts with the resulting PMNPs.

### 6.12 Albumin-Coated PMNP

Protocols for preparation of drug loaded albumin coated paramagnetic nanoparticles (alb-PMNP):

### Stock Solutions:

a. 50 mg of paramagnetic nanoparticles (PMNPs) are washed and suspended in 40 ml of a 10% ethanol in methanol solution. The mix is then sonicated to create a homogeneous dispersion.
b. A stock solution of the to be loaded drug (e.g. adriamycin, curcumin, taxol) is prepared in 10 ml of methanol
c. A solution of albumin (either HAS or BSA) is prepared by dissolving the albumin in DI water (5 mg/ml).
d. A solution of PEG (typically either methoxy PEG-DSPE, Alexafluor-750 conjugated to PEG-DSPE or other PEG-DSPE products with any of several standard fluorophores covalently attached) is prepared: 1 mg/1 ml in DMSO.
e. A dye solution for bioluminescent imaging.

Procedure 1: A homogeneous solution of (a) is mixed with (b) while sonicating continuously thus generating a homogeneous suspension of the drug plus PMNP mixture. Solution (c) is then added in drops with sonication thereby creating a silky suspension. The protocol can be tuned to vary the amount of albumin coating the PMNPs. Fluorescence from albumin and/or drug can be used to determine the amount bound to the PMNP by monitoring the albumin/drug fluorescence in the PMNP free solution after either spinning down the PMNPs or using a strong magnetic field to collect the PMNPs. The final particles size (reflecting the amounts of albumin associated with the PMNPs) can be tuned by varying the relative alcohol concentration (in step a).

The resulting PMNPs are separated from unbound molecules (albumin, drug, drug loaded albumin) by placing a high power magnetic field next to the vessel containing the suspension and allowing the magnetic separation to proceed overnight. The PMNP free solution can then be separated from the PMNP's that have accumulated by the magnetic field. The PMNP's can be washed and re-separated as deemed necessary. The result of the separation process is a suspension that contains drug loaded alb-PMNPs with minimal amounts of drug not associated with the PMNPs. For extended circulation in the animals/humans the resulting drug loaded alb-PMNPs are treated with known amount of solution (d) using the methoxy PEG-DSPE. If the alb-PMNPs are to be used for fluorescence imaging studies, then instead of using methoxy PEG-DSPE, we use either a commercially available fluorescence-labeled PEG-DSPE, (e.g. Alexafluor-750) or a derivatized (e.g. maleimide, amine) PEG-DSP to which we conjugate an appropriate fluorophore (using step e). The final particles were washed with DI water and lyophilized overnight to get dry drug loaded alb-PMNPs.

Procedure 2: The procedure can be easily modified to accommodate any commercially available drug-albumin conjugate and drug loaded albumin nanoparticles (e.g. Abraxane, which a widely taxol conjugated albumin). These materials can be directly conjugated to PMNPs by mixing with buffer washed PMNPs, collecting the coated PMNPs (as described above) and then lyophilizing overnight to get dry drug loaded alb-PMNPs.

Results indicate that very effective binding of all the above reagents and drugs to the PMNP is observed; fluorescence labeled abraxane-coated PMNPs have been shown to rapidly localize in the pancreatic tumors and metastatic lesions in the liver; IV infused curcumin-loaded alb-PMNPs have been shown to target glioblastomas in the brains of mice and inhibit their growth; albumin-PMNPs appear to cross the blood brain barrier; and alb-PMNPs will accumulate in tumors even without the use of the external magnetic field though much less efficiently and much more slowly.

### 6.13 MRI Imaging of Magnetic Field Induced Localization of Gadolinium Oxide Based PMNPs in Tumors

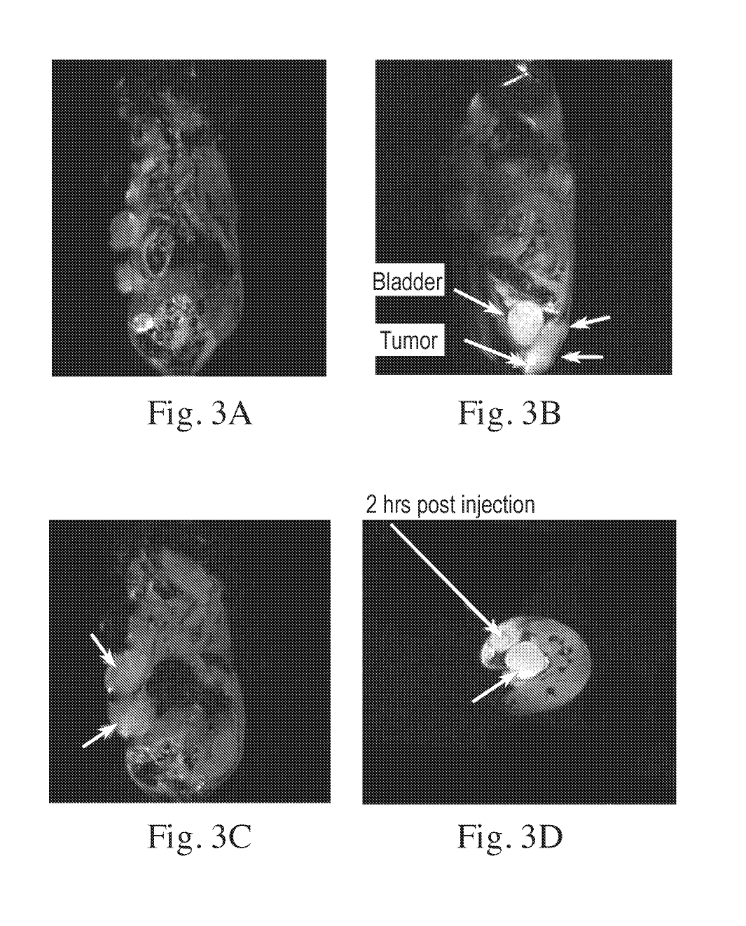

In FIG. 3 are presented results on magnetic immobilization of gadolinium-based PMNPs in a mouse with numerous human breast cancer xenographs. The MRI taken immediately after injection with PMNPs is shown in FIG. 3A. There is no image enhancement for any of the multiple tumors located in different parts of the mouse including one at hind end (green-arrow at bottom of the picture marked tumor). The mouse was then removed and then subjected to thirty minutes of having a magnetic field placed over the tumor demarked by the green arrow. FIG. 3B shows one of the many very similar MRI images acquired after the removal of the magnetic field for a period of hours. The intensity of MRI signal is very similar at both tumor and bladder. This is very different than observed in case of MAGNEVIST where there is a transient image of the bladder and no site specific localization of the contrast agent in any one of the tumors.

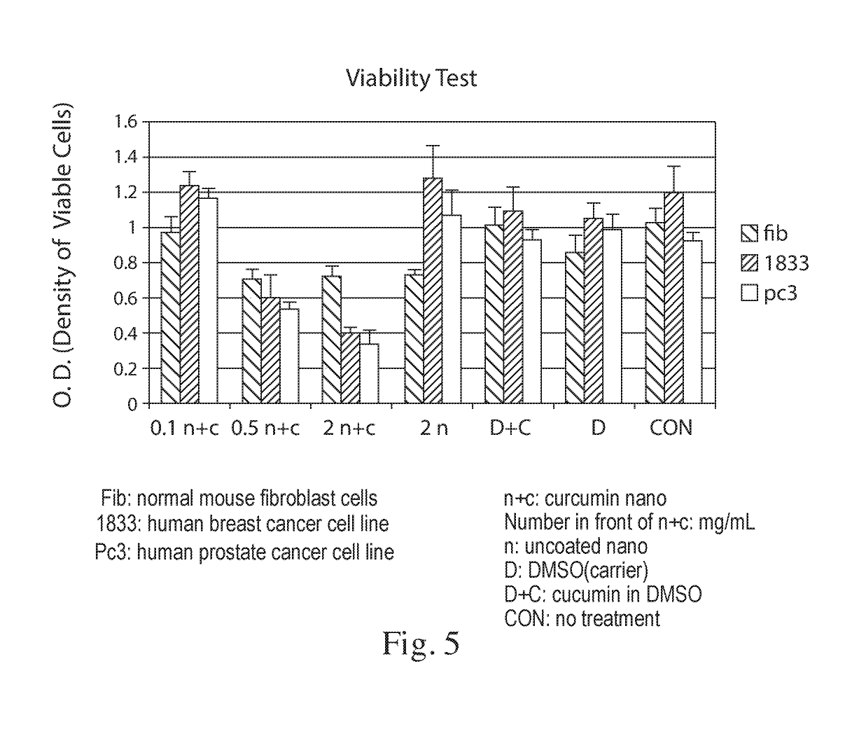

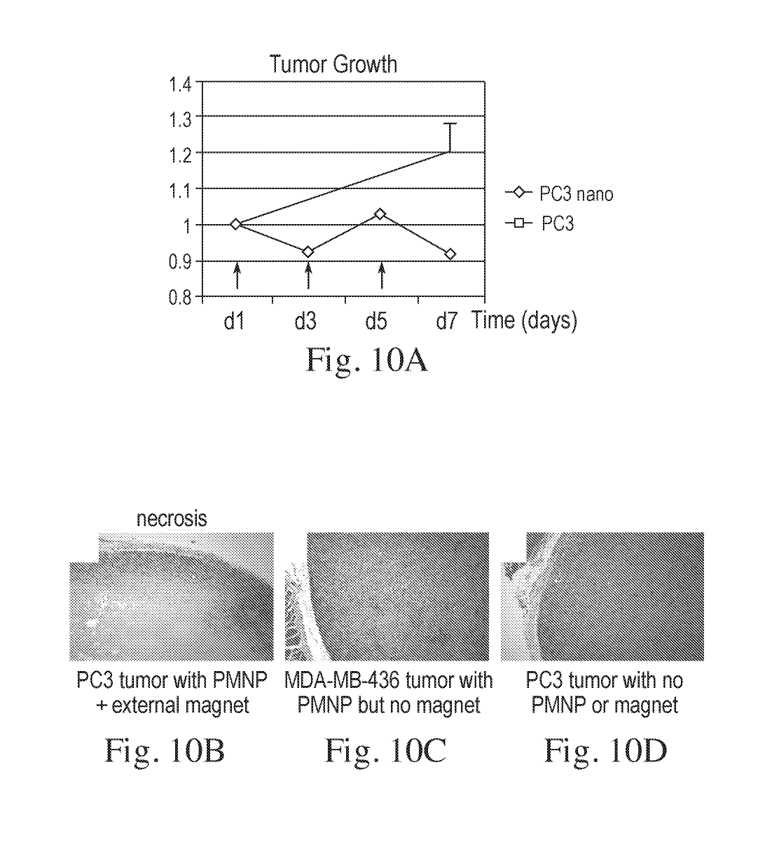

FIG. 3C-13D shows similar results for another mouse with human breast xenographs. The PMNPs are concentrated at site of a lower abdominal mammary tumor (shown in FIG. 3C) using an external magnetic field placed over the tumor for 30 minutes. The contrast increased significantly (FIG. 3D) subsequent to treatment with the magnetic field. Imaging was performed over several hours without taking the mouse out of MRI machine thereby maintaining all of the imaging parameters. Enhanced images of the magnetic field targeted tumor site were observed as well as in the bladder which demonstrates efficient excretion of PMNPs that are not attached to tumor.

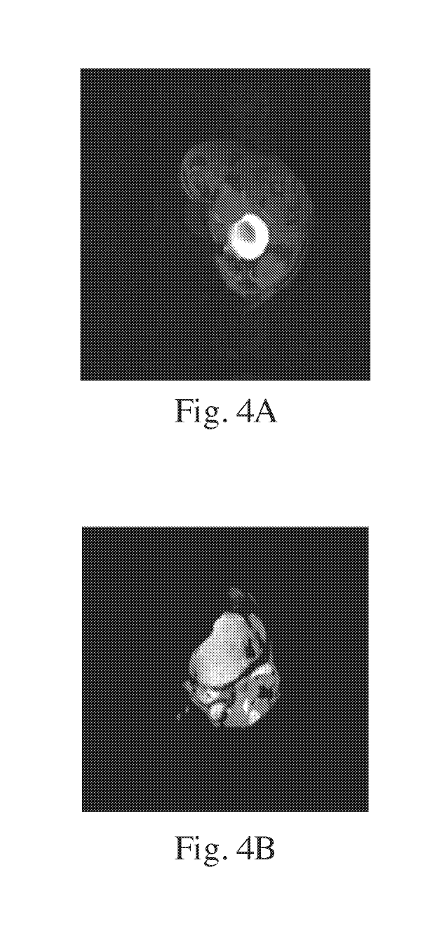

A comparison of MRI signal between MAGNEVIST (the most common MRI contrast agent) and the PMNPs was performed. Within a short time virtually all the MAGNEVIST concentrates in the bladder with high MRI signal intensity as shown in FIG. 4A (bladder). In the next FIG. 4B is shown the MRI from gadolinium oxide-based PMNPs that were injected and then localized at a tumor site using a magnetic field. The signal intensity at the tumor site was comparable to the signal intensity generated by MAGNEVIST at the bladder (without application of magnetic field).

### 6.14 Therapeutic Effect of Curcumin-Loaded OA-PMNPS on Tumor Cells In Vitro and In Vivo

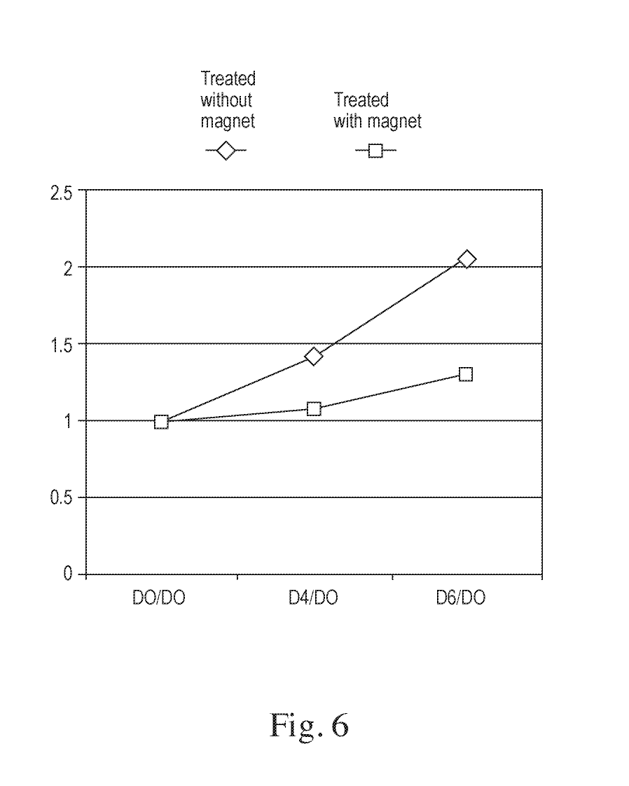

Curcumin-loaded OA-PMNPs are effective at killing tumor cells, as shown in FIG. 6. Viability test for three different types of cells, fibroblasts from normal mice and two cancer cell lines, treated with various concentrations of curcumin loaded OA-PMNP (gadolinium oxide based) nanoparticle (n+c) as well as several controls including uncoated curcumin (c), carrier DMSO (D) and uncoated OA-PMNP (n). These cells were incubated for 24 hrs in a 96-well culture dish and then the amount of viable cells in each well was measured using MTT test. Higher OD in the graph indicates higher density of viable cells. The results indicate efficacy for the curcumin loaded OA-PMNPs with respect to killing of tumor cells.

A 100 μL, injection of curcumin loaded OA-PMNPs was given to two mice each with breast cancer xenographs (MDA-MB-436). In one mouse, an obvious tumor was exposed to a magnetic field for 45 minutes whereas no magnetic field was placed on the other mouse. A significant difference in the tumor-growth rates were observed over 6 days.

Synergy between curcumin and Adriamycin: A single mouse with breast cancer xenograph tumors was pretreated with curcumin-loaded OA-PMNPs and then treated with adriamycin-loaded OA-PMNPs. The magnetic field targeted tumor underwent regression much faster than similar tumors treated exclusively with adriamycin-loaded OA-PMNPs.

Accordingly, a new biocompatible MRI-active paramagnetic nanoparticle platform has been developed for both targeted imaging and targeted drug delivery. The paramagnetic nanoparticles (PMNPs), preferably with a core of europium-doped gadolinium oxide nano-crystals, can be easily and heavily coated with either individual drugs or combinations thereof. This versatility offers a new approach to PMNP exploitation, using hierarchical targeting that couples magnetic targeting to lesion macrodomains with ligand-directed targeting to critical tissue components (microdomains). Using the new platform, results show: i) prolonged magnetic field-induced localization of the particles in mice as seen in magnetic resonance imaging (MRI); and ii) dramatic regression for magnetic field-targeted tumors in mice subsequent to infusion with Adriamycin-coated PMNPs. Extension of this to specific target sites is applicable. Targeting tumor cells in bone metastases can be effected, for example by: 1) attaching CXCR4 antagonistic peptide for anchoring of PMNP onto tumor cell surfaces; and 2) integrating inhibitors to target the oncogenic Src pathway. Targeting tumor-supportive host cells in bone metastases can be effected by: 1) adjoining isphosphonates for PNP targeting of tumor-associated osteoclasts; and 2) packing cfms (CSF-1R) inhibitors for targeting tumor-associated macrophages. MicroPET/CT can be used to quantitatively measure the inhibition of tumor outgrowth in bone and the recovery of tumor-derived bone lesions.

For the technique of macro-localization and micro-localization, magnetic delivery first directs the particles to the macro-domain of the lesion, and specific targeting molecules bound to the particles then focus drug delivery to specific cellular components or pathways within the lesional microenvironment. In contrast to common strategies currently used for molecular targeting with non-magnetic particles (12, 13, 14), the targeting ligands in this platform (for example, the CXCR4 antagonist) are not be used as a ‘seeker’ to search for tumor cells in the entire body, but instead either as a local ‘anchor’ to secure the particle onto specific cells (for example, tumor or normal host cells) at the lesion or alternatively as a pathway inhibitor or cytotoxin whose domain of action has been focused by magnetic localization. Consequently, the ‘burden’ of specificity for the peptide is greatly reduced, and the efficiency of targeting is enhanced. In addition to increased efficiency, the combination of magnetic and ligand-based targeting is expected to reduce off-target ligand effects by orders of magnitude, an area of critical need in reducing toxicity and raising therapeutic index. For example, the targeting of osteoclasts in metastatic bone lesions is limited by drug effects on normal hematopoietic tissue (8, 15). Since few pathways are completely specific to lesional cells, regional localization by magnetic field offers a uniquely valuable synergistic element to ligand-based approaches. Unique coatings for europium doped-gadolinium oxide (Gd₂O₃/Eu)-based paramagnetic nanoparticles to support multiple targeting. The several distinct strategies for high density incorporation of various types of targeting molecules on our paramagnetic nanoparticles described herein may be employed in these methods. Individual particles can carry multiple therapeutic drugs in addition to targeting peptides, enhancing the versatility of therapeutic strategies. Multiple-target designs permit simultaneous targeting of several contributing pathogenic cell types, such as the tumor and non-tumor cells that interact in metastatic lesions. In addition, the Gd₂O₃-based platform readily accommodates surface decoration with: i) radioactive iodine labeled peptides for μPET imaging; ii) oleic acid for facile incorporation of hydrophobic drugs and targeting molecules; and iii) PEG and other polymeric species for modification of the circulatory dynamics and delivery properties of the nanoparticles. This degree of multi-functional plasticity for paramagnetic nanoparticles has not been reported.

Results show that magnetic fieldic localization of the novel PMNPs can be utilized to: i) image via MRI the extent and duration of magnetic localization; and ii) target the delivery of therapeutic drugs to tumors resulting in an improved efficacy/potency with a concomitant reduction in systemic toxicity.

### 6.15 Drug Delivery to Tumors by Magnetically Localized PMNP

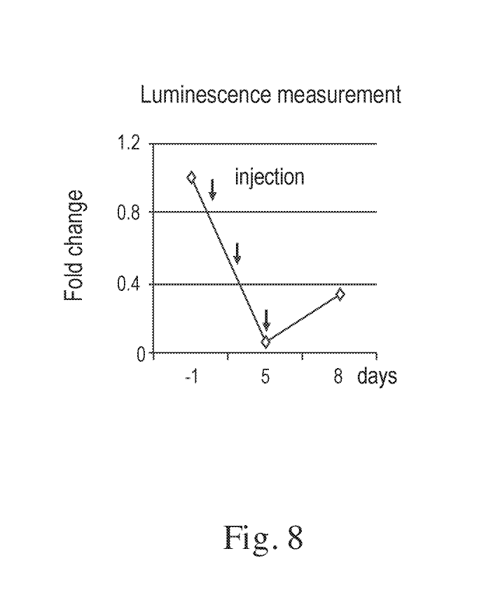

PMNP-bound adriamycin (ADM) has superior anti-tumor efficacy and potency relative to free drug and that efficacy is correlated with both PNP localization and local drug delivery. (FIG. 8) A biocompatible coated PMNP have been synthesized based on europium-doped gadolinium oxide nano-crystals (Gd₂O₃:Eu). With an oleic acid coating and capping, the new particle has a high affinity for hydrophobic drugs such as adriamycin, curcumin, taxol and others. The strong paramagnetism of these PMNP supports facile localization of infused particles by applying a magnetic field over the targeted tissues. The non-toxic form of gadolinium in the Gd₂O₃:Eu is a strong contrast agent for MRI, facilitating the use of non-invasive imaging to monitor PNP localization. The PMNP are stable, in certain embodiments, with size ranges of 50-60, 60-80 nm, 80-100 nm, 100-150 nm and 150-200 nm and remain in suspension for many days without aggregation.

### 6.16 Magnetic Field-Directed Localization of PMNP was Examined in a Mammary Tumor Model (PyMT Mice)

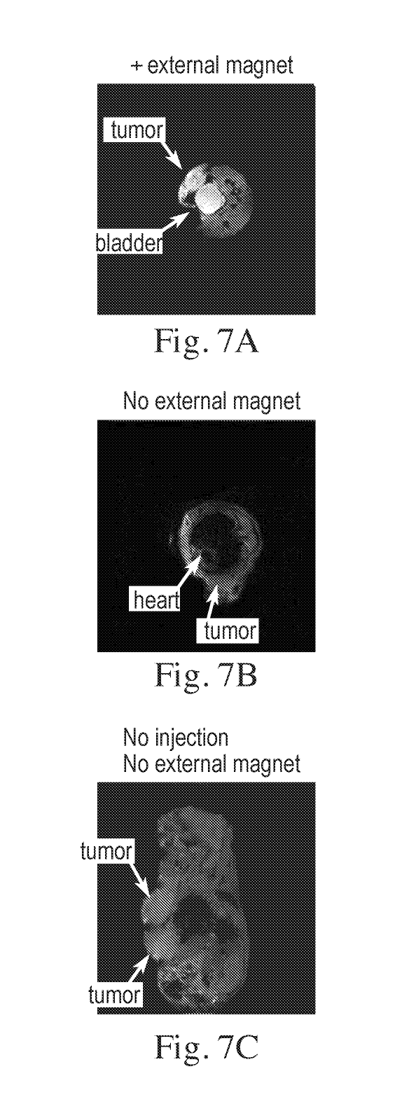

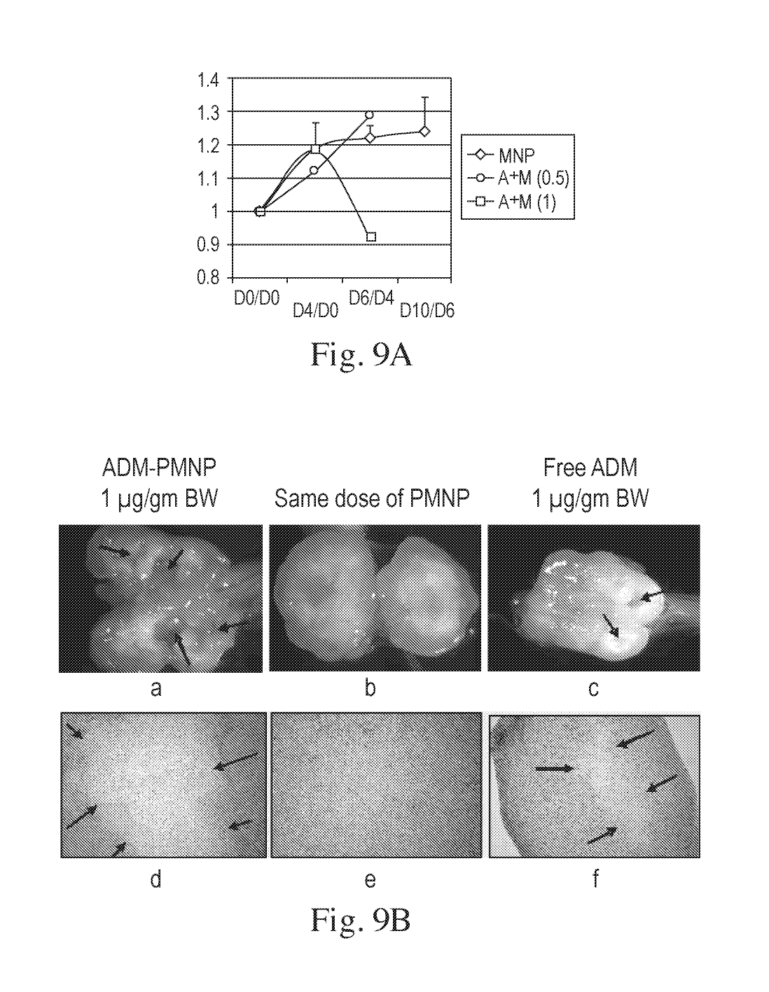

A strong MRI signal, indicating the accumulation of PMNP, was detected in magnetic field-treated but not untreated tumor (FIG. 7). Signal intensity in treated tumor was similar to that in bladder, a major route of PMNP elimination. Next, to assess drug delivery and anti-tumor efficacy, MoB-1833 mammary xenografted tumor-bearing mice were infused with ADM-coated PMNP (ADM-PMNP). ADM use in chemotherapy is limited due to systemic toxicity and it is therefore an excellent model drug for evaluation of the potentially utility of our PMNP platform. FIG. 9 shows results for a mouse treated with ADM-PMNP (1.0 mg ADM/kg body weight/dose) three times at two day intervals. This ADM dose is 5-20 times lower than doses of free ADM commonly used for in animal studies and similar to doses used clinically. The 4-day treatment achieved a 9-fold reduction in tumor mass (FIG. 8). Tumor rebound upon drug termination confirmed specificity of the drug effect. These data were confirmed using a different human breast cancer line, MDA-MB-436 (FIG. 9A). Consistent with drug effect on tumor growth, multiple large necrotic pits were observed in magnetic field-exposed, ADM-PMNP-treated tumors, but not in magnetic field-exposed tumors treated with unmodified PMNP (FIG. 9B a vs.b). In comparison, smaller necrotic lesions were seen in tumors treated with free ADM (1.0 mg/kg) in the absence of PMNP. The necrosis was confirmed histologically (FIG. 9Bd-f). Similar effects of ADM-PMNP on tumor growth and tumor necrosis were observed using xenografted human prostate cancer cell line, PC3 (FIG. 9C and data not shown). These results, in a limited number of mice, indicate that this new platform is able to concentrate therapeutic drugs site-specifically in vivo.

### 6.17 A Histopathological Analysis was Performed on Multiple Tissues from these Mice to Obtain a Preliminary Assessment of Toxicity

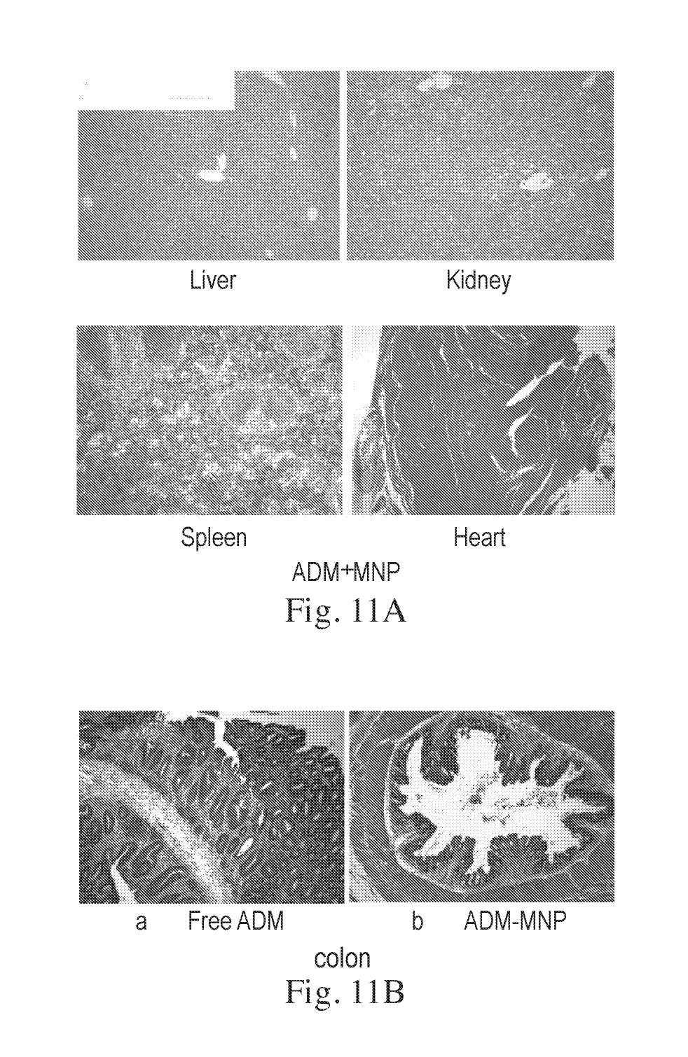

No obvious abnormalities were observed with ADM-PMNP/magnetic field treatment, except a mild change in spleen initially suggesting extramedullary hematopoiesis which in a subsequent more extensive histopathology study including 5 animals studied over three weeks of ADM-PMNP infusions turned out to within the bounds of normal spleen tissue (FIG. 11A). In comparison, colonic inflammation and hyperplasia were observed in several mice treated with free ADM but not in ADM-PMNP-treated mice (FIG. 11Ba vs.b), indicating the new platform may reduce systemic toxicity in intestine, a common toxic effect in clinical chemotherapy (1-4).

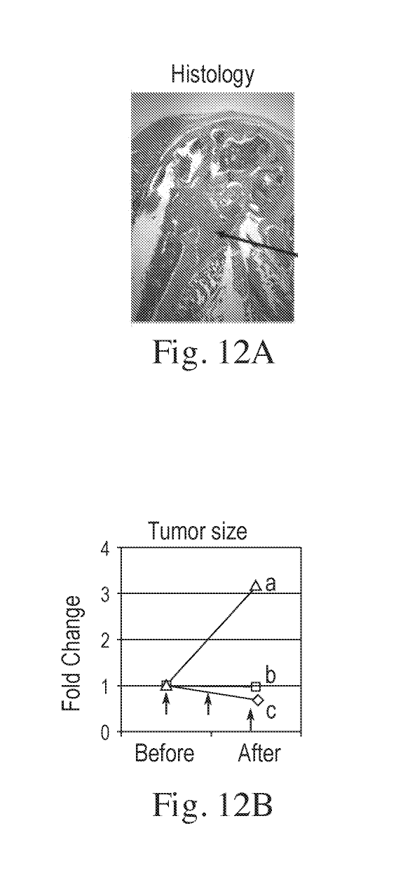

These studies using the bone metastasis model confirm its appropriateness. As previously reported (6, 7), cardiac inoculation of BoM-1833 cells into nude mice led to the development of multiple bone and brain metastases that were detected by bioluminescent imaging. Treatment with ADM-PMNP, followed by magnetic field application at a single tumor site, resulted in a decrease in the size of the magnetic field-exposed tumor but not the other tumors in the animal (FIG. 12B). The tumor location within the bone marrow cavity of the right knee was confirmed by histological analysis and data not shown). Two additional nude mice carrying BoM-1833 bone lesions were tested using the same procedure, and reduced tumor growth was again observed for magnetic field-exposed compared to untreated lesions (data not shown). These results are consistent with an anti-tumor effect dependent on magnetically localized ADM-PMNP.

To increase specificity and efficacy, two strategies can be used to target tumor cells in bone metastases. The first is an anchoring strategy based on tumor expression of the cell surface chemokine receptor, CXCR4. The second is a signal transduction targeting strategy based on tumor cell activation of the Src kinase pathway. The Src pathway has been found to play an important role in tumorigenesis and metastasis and therefore is an important therapeutic target of many solid tumors (8). Previous studies have demonstrated that Src activation is associated with bone metastasis in human breast cancer and the activation of this pathway was found to be critical for the survival and outgrowth of BoM-1833 cells in bone metastases (9). Similar to CXCR4, described below, Src pathway is also actively involved in the function and growth of various normal cells/tissues (10, 11).

### 6.18 Anchoring Strategy

This strategy targets tumor cells via the chemokine receptor CXCR4. CXCR4 is expressed at high levels in the BoM-1833 cells used in the model and is also strongly expressed in a majority of human breast cancer bone metastases (6, 9). While CXCR4 is a locally specific marker for tumor, it is relatively non-specific from a whole-body perspective, as it is expressed in numerous tissues (12). Consequently, a large gain in specificity is achievable by the use of this target in conjunction with magnetic localization. CXCR4 is therefore an appropriate molecule with which to test the concept of hierarchical targeting. In one embodiment of the method, a small peptide CXCR4 antagonist, CTCE-9908, binds both murine and human CXCR4 and inhibits the invasion and growth of CXCR4-positive tumor cells (13, 14). This peptide (designated 9908), or its scrambled control (S9908), is attached to the PMNPs. Several strategies are available for peptide-PMNP linking. One is to attach peptide to one end of a bifunctional polyethyleneglycol chain, whose other end contains a maleimide group that can be bound to reactive thiols incorporated into silane derivatives that coat the Gd₂O₃/Eu core. This laboratory has found that a silane coating does not interfere with subsequent coating with oleic acid, permitting the further incorporation of hydrophobic drugs such as ADM and the other drugs and dyes.

Once the anchoring and control peptides are attached to PNP, various cell lines, including BoM-1833 cells, other CXCR4-positive cancer cells, and CXCR4-negative CHO cells ¹⁵ are used to assay the efficacy of binding to CXCR4⁺ cells and retention of specific binding after further coating of PNP with oleic acid.

### 6.19 In Vitro-Validated 9908-PNP is Tested In Vivo Using the BoM-1833 Xenograft Model

Control mice receive 59908-PMNP or PMNP without peptide. (For structure of 9908 see Hassan et al, International Journal of Cancer Volume 129, Issue 1, pages 225-232, 1 Jul. 2011, hereby incorporated by reference). In each of these three treatment groups, half of the mice have magnetic fields applied to tumor sites. Three endpoints are collected: 1) efficiency of localization to tumor sites, assessed by MRI as in FIG. 7; 2) duration of PNP residence at tumor sites during a 1 wk period following magnetic field removal (assessed by MRI); 3) effect on tumor size, assessed by bioluminescence. Magnetic localization is quantified by subtracting the localization observed in the absence of magnetic field exposure. 9908-PMNP and 59908-PMNP can each be prepared with and without incorporation of ADM, and the four PMNPs are administered to mice with bone metastasis.

The efficiency and/or duration of magnetic localization will be enhanced for 9908-PMNP compared to other PMNP, and anti-tumor efficacy against bone metastasis will be enhanced for ADM-9908-PMNP treatment compared to ADM-PMNP treatment. Alternative designs include PMNP coupling to anti-CXCR4 antibody or targeting to other highly expressed tumor cell surface markers, such as TGFβ receptor or Mud (12).

### 6.20 Targeting the Src Pathway

Activation of the Src kinase signaling is associated with bone metastasis in human breast cancer, and the activation of this pathway is critical for the survival and outgrowth of BoM-1833 cells in bone metastases (9). This pathway is therefore an excellent candidate for targeting of PMNP. As with CXCR4, the Src pathway is important for the function and growth of various normal cells and tissues (10, 11), and therefore provides a similar rationale for enhanced specificity via magnetic localization.

Among several Src inhibitors under development, Dasatinib is the most supported by clinical studies in a variety of solid tumors (8, 16). However, systemic administration of the drug is associated with multiple toxic side effects (8). In addition to tumor cells, Dasatinib was found to inhibit the activity of osteoclasts, the host cells that play a crucial role in the development of tumor-associated bone lesions (17, 18). Dasatinib, like ADM, is a hydrophobic molecule, and we will employ our established approach to attach it to PMNP via an oleic acid coat. After attachment is achieved, of Dasatinib-PNP to inhibit tumor cell growth and survival is assessed in vitro using a variety of human cancer cell lines, including BoM-1833. The procedure outlined herein is used to examine its anti-tumor function upon magnetic localization in vivo, using both the xenograft and bone metastasis models. Control groups are untreated or treated with free drug or ADP-PMNP.

The ability of Dasatinib to interact with ADM or the 9908 peptide in the PMNP platform is examined, including whether Dasatinib sensitizes tumor cells to ADM.

Efficacy of PNP tailored for targeting of tumor-supportive osteoclasts and macrophages. Recent studies demonstrate a key tumor-supportive role for non-tumor host cells, especially cells of myeloid lineage (2-7). The interaction between tumor cells and the bone microenvironment, especially host osteoclasts, is termed a ‘vicious cycle’ and plays a critical role in the formation of bone metastases (8,9,10). Macrophages in the bone microenvironment also participate in the formation of tumor metastases (11, 12). Consistent with these findings, a marked infiltration of osteoclasts and macrophages in BoM-1833-induced bone lesions (has been observed herein data not shown).

Targeting tumor-associated osteoclasts in the bone microenvironment: Bisphosphonates (BPP) are potent inhibitors of osteoclast-mediated bone resorption and their role in the treatment of patients with metastatic skeletal diseases is well established (13). However, BPP treatment is associated with multiple systemic toxicities, including osteonecrosis of the jaw, hypocalcaemia, and gastrointestinal toxicity (14, 15, 16). Developing BPP-PMNP as an effective and less toxic alternative to BPP, and also targeting a lesional component distinct from that targeted by drugs such as ADM, can add therapeutic value in the context of multi-targeting PMNP.

To incorporate BPP into the PMNP platform, the long side chain found in certain BPP (pamidronate, neridornate, and alendronate) can be utilized, which provides a convenient primary amine structure that can be attached to maleimide groups on bifunctional polyethyleneglycol precoated on PMNP. As in the case of peptide attachment, this strategy allows additional incorporation of other therapeutic drugs into the same PMNP via secondary oleic acid coating. The ability of BPP-PNP to induce apoptosis of osteoclasts can be determined in vitro in primary myeloid cultures (17, 18). MTT and TUNEL assays can be used to compare the effect of BPP-PNP to free BPP and uncoated PNP on osteoclasts. If positive results are obtained, we will examine the impact of BPP-PNP on BoM-1833-induced bone lesions using endpoints that focus on potential benefits for tumor-associated bone structure, as follows: 1) MRI imaging can be combined with bioluminescent imaging in order to localize the bone lesion and determine if BPP-PNP can be localized into the lesion by magnetic field; and 2) μCT can be combined with bioluminescent imaging to assess bone damage and examine the efficacy of BPP-PNP in preventing or ameliorating bond defects.

Targeting tumor-associated macrophages in the bone microenvironment. The macrophage growth factor CSF-1, acting through its receptor, c-fms, plays a critical role in the development of breast cancer bone lesions (10, 19). It has been demonstrated that CSF-1 is a crucial facilitator of tumor progression and metastasis (6, 20). Incorporating c-fms inhibitors into the PMNP platform can be effected to inhibit tumor-associated macrophages in bone metastases. Several c-fms inhibitors are available commercially. Gw-2580 is able to block multiple CSF-1-induced activities both in vitro and in vivo (21), and CYC10268 can repress pro-inflammatory cytokine production from murine macrophages (22). Both are hydrophobic molecules and can be incorporated in high density in the oleic acid-coated PMNPs described herein.

### 6.21 Nitric Oxide has Tremendous Therapeutic Potential

Systemic delivery of large doses of NO is limited in that excess NO can result in hypotensive shock. The nanoparticle platform method described here allows for delivery of NO to sites that are targeted by an external magnetic field which localized the infused nanoparticles at the site defined by the externally applied magnetic field. Using this approach, localized tissues that suffer from poor blood perfusion (e.g. many types of tumors as well tissues compromised due to vascular disease or damage) can be targeted and experience enhanced NO-induced blood flow allowing an increase in delivery of oxygen, nutrients and drugs. Additionally in many types of tumors, NO can be cytotoxic. This approach results in targeted delivery of cytotoxic levels of NO to tumors. An embodiment allows for simultaneous delivery of drug (loaded on the nanoparticle) and increased tissue perfusion via the same localized nanoparticle.

The objective is to develop a nitric oxide delivery vehicle suitable to localized delivery of NO without creating a potentially deleterious systemic overload of NO. The strategy employed in the presented platform is to coat paramagnetic nanoparticles with reactive thiol groups that can then be converted into S-nitrosothiols. S-nitrosothiols are relatively stable compared to NO and perform most of the same therapeutically relevant functionalities as NO by transferring the thiol bound NO to thiols on other thiol containing molecules. S-nitrosothiols are highly effective as vasodilators and anti-inflammatory agents. The development of paramagnetic nanoparticles that can actively transfer NO via S-transnitrosation will allow for magnetic targeting of tissues that require the enhanced local introduction of NO bioactivity as part of a therapeutic regimen.

Protocols: Two step process for S-nitrosation of paramagnetic nanoparticles (PMNP):

i). Introduction of reactive thiols on the surface of the PMNP; and
ii). S-nitrosation (creation of an SNO group through the addition or transfer of a nitrosonium ion, NO+ to the reactive SH/S− group of the thiol) of the introduced reactive thiols on the surface of the PMNP.

In a variation on the above, the thiol-containing reagent is first S-nitrosated and then attached to the PMNP.

The PMNP's can be a gadolinium oxide based nano-crystal with or without dopants such as Eu added to enhance the paramagnetism or luminescence properties. The PMNP's used in this preparation are based on Gd₂O₃ nanocrystals. The coatings used to create the NO loaded PMNP are general and not specific to any one kind of core PMNP. The use of Gd based PMNP core also allows for a positive contrast MRI imaging agent.

Methods: Step i: Thiolation of the PMNPs:

Method 1. Thioglycolic acid (TGA) as the thiolating agent: 10 mg of the PMNP (paramagnetic nanoparticles) are washed several times with tris(HCl) 50 mM pH 7.5, followed by another multiple washings with a solution of TGA (200 mM) in the tris buffer pH 7.5. The TGA (thioglycolic acid) is strongly adsorbed on the surface of the PMNPs as seen by labeling the thiols and spinning down the PMNPs-all the color is in the spun down PMNPs.

Method 2. Use of dimercaptosuccinic acid (DMSA) to introduce two thiols per coating molecule. DMSA has two thiols and two carboxylic acids. Carboxylic acid binds very tightly to the surface of the gadolinium oxide particles. 100 mg of PMNP was dispersed in 10 ml of chloroform followed by addition of 50 μL triethylamine. 50 mg of DMSA was dissolved in 10 ml dimethyl sulfoxide (DMSO) and then mixed with the above solution/suspension containing the PMNP. The resulting solution was vortexed at 600 C for 4 hrs. The turbid PMNP suspension was then centrifuged to collect the particles. The PMNPs were then washed with ethanol and DI water. This entire process can be repeated to further enhance coverage of the PMNP with DMSA.

Method 3. Use of 3-mercaptopropyl-trimethoxysilane (3 MPTS) as the source of reactive thiols. 10 mg of PMNPs were washed several times with deionized water many times, centrifuged and collected. 20 μl of 3 MPTS was added to the above particles and sonicated for 10 min and washed quickly. The coated particles were washed several times with deionized water followed by tris(HCl) 50 mM pH 7.5.

Method 4. Use of a bifunctional PEG with one end containing either a carboxyl or phospholipid group (both of which anchor the PEG to the surface of the PMNP) and the other end containing a thiol. The advantage of this approach is that the PEG can be anchored on to a oleic acid coated PMNP that can function as a transported of lipophilic drugs such as Adriamycin, taxol, curcumin.

Method 5. A variation on the all above methods entails first S-nitrosating the thiols and then attaching them to the PMNP. The following protocol was used to prepare SNO-PMNPs for the in vivo measurements described below. Either 1 mg/ml of DMSA or 20 microliters of MPTS in 50 mM tris HCl buffer pH7.5 is titrated with NO gas in the presence of oxygen until it forms the pink solution. The resulting pink solution is treated with buffer-washed PMNPs (10 mg) over 24 hrs in dark. The resulting pink PNMPS were spun down and washed with buffer and collected as wet particles and then used for the in vivo experiments. The PMNP's are re-suspended in buffer prior to infusion.

Step ii: Nitrosothiol formation (SNO) formation for the coated PMNPs:

10 mg of the either of the two thiolated PMNPs from the above methods were washed with PBS 50 mM pH 7.5 buffer and treated with 5 mM DTT (dithiothreitol) in PBS 50 mM pH 7.5 buffer to reduce any disulfide bonds and thus maximize the number of available reactive thiols. The PMNPs were washed with buffer to get rid of excess of DTT and treated with buffer saturated with pure NO gas (from either a gas tank or from NONOates). The particles turned pink indicating that the PMNP-SNO species has formed. PMNP-SNO particles were washed and stored in dark at 40° C.

In vitro results: All of the above protocols yielded SNO labeled PMNPs as reflected in the bright pink colored particles that were spun down in a centrifuge.

In vivo results: Positive results were obtained using the method where the reagent is first S-nitrostated. Both products were tested and yielded similar results.

Reducing localized reperfusion injury through the use of magnetic field targeted NO delivery in hamster model. Paramagnetic NOnp with a magnetic field applied: increased reperfusion; accelerated the recovery of FCD; reduced leukocyte adhesion; reduced apoptosis and necrosis. The combined approaches appear especially effective. Similar benefits are obtained by targeting NO to the site of an induced coronary blockage in a hamster model. Targeted NO delivery by exposing to magnetic field using this platform inhibited the physiological changes that are associated with cardiogenic shock (e.g maintained cardiac tissue perfusion, FCD and minimized indications of inflammation.

### 6.22 Chemicals and Materials

The following chemicals were obtained from Sigma-Aldrich Chemical Company (St. Louis, Mo.): bovine serum albumin (BSA-30% solution), penicillin-streptomycin solution, trypsin-EDTA solution and fibronectin. Fetal bovine serum (FBS) was purchased from Hyclone Laboratories (Logan, Utah). Dubelco's Modified Essential Medium DMEM, phenol red-free DMEM and Dubelco's modified phosphate buffered saline DPBS from Cellgro (Manassas, Va.). Astrocyte Medium was purchased from Sciencell (Carlsbad, Calif.). Transwell polyester filters (12-mm diameter, 0.4-μm pore size) were purchased from BD-Falcon (San Jose, Calif.).

Primary polyclonal anti-Von Willebrand Factor (VWF) 14014 was purchased from Santa Cruz Biotechnology (Dallas, Tex.) and monoclonal anti Glial fibrillary acidic protein (GFAP) G3893 was purchased from Sigma-Aldrich. Matching secondary antibodies and CellTracker CM-DiI were purchased from Molecular probes (Carlsbad, Calif.).

Paramagnetic (10-100 nm) Gd₂O₃ nanoparticles were purchased from US research Nanomaterials Inc. (Houston, Tex.) and coated according to specific protocols developed at Einstein.

### 6.23 Nanoparticles Synthesis and Conjugation

Two nanoparticle platforms were fabricated, the first one uses variations on a hybrid sol-gel/glass (sugar derived) nanoparticle platform process that yields highly stable biocompatible nanoparticles. This process allows for a homogeneous size distribution (radius of 80 to 200 nm depending on method of preparation), tuning of rates of drug release, easily modifiable surface (including ligands for tissue targeting), extended circulation time, variable type and amount of matrix and stability with respect to pH and temperature. Most significant is the ability to control matrix release rates over very large time windows and chemical modification of charge and surface coatings. The systemic delivery of these NPs is achievable via intravenous or intra-peritoneal injection but enhanced transdermal delivery or sublingual delivery with agents to open skin pores are also feasible. The second nanoparticle drug delivery platform is based on paramagnetic Gd₂O₃ nanocrystal NPs with coatings developed at Einstein that allow for loading with curcumin as well as other potential chemotherapeutics such as Adriamycin. These NPs are highly paramagnetic allowing for the use of an external magnetic fieldic field to localize them at a targeted site. Additionally, the Einstein team has developed a straightforward method of conjugating PEG chains to the surface of the NPs (patent pending) which allows for extended circulation time, cell uptake properties and attachment of targeting molecules such as peptides, aptamers and antibodies. Attachment of fluorescent probes to either platform has proven straightforward.

NPS were synthesized by attrition. Primarily 600 μl of 1 mM HCl were added to 3 mL of Tetramethyl orthosilicate (TMOS). This solution was then sonicated on ice for 15 minutes until a single phase was acquired. The hydrolysis of TMOS resulted in the formation of Silicone dioxide (SiO2) and Methanol (MeOH). The hydrolyzed TMOS was left in ice for another 15 minutes. The primary 50 mM phosphate buffers for each of the samples differed in their MeOH concentrations (1: 70%, 2: 80%, or 3: 90%). The more MeOH molecules there were in the primary buffer, the smaller the size of the NPS, because MeOH molecules inhibit the formation of hydrogen bonds between adjacent SiO2 molecules. The secondary buffers consisted of 1.5 ml PEG 400 and 1.5 ml Chitosan. After the secondary buffers were added to the primary buffer, 600 μl of N-(2-aminoethyl)-11-amino-undecyltrimethoxysilane (AUTS) were added to one batch of NPS The AUTS carried two amino groups which subsequently added positive charges to the NPS. To confer fluorescence, 30 μl of Alexa-488 were added to all NPS.

### 6.24 Nanoparticles Analysis

Once the nanoparticles were synthesized, the characterization of their physical properties (particle size, dispersion stability) was performed by dynamic light scattering (DLS) and the NP coatings were monitored by absorption (using the Perkin Elmor UV/Vis spectrophotometer) where fluorescence was determined using a PTI steady state/nanosec time-resolved spectrofluorimeter, and a Nikon fluorescence high resolution microscope.

### 6.25 Cell Culture

An in-vitro model of the BBB was developed by growing mouse brain endothelial cells (bend.3) form ATCC (Manassas, Va.) or a co-culture of these cells with immortalized astrocytic cell line. The line of immortalized wild type cortical astrocyte (IWCA) was originated as follows; normal mouse cortical astrocytes cells were obtained from E19 pups and cultured in astrocyte medium (DMEM 1 g/L glucose). Medium was changed every 3 days and the cells were cultured for 10 to 14 days until confluence. At this point the cells were immortalized using human telomerase reverse transcriptase (hTERT). hTERT cDNA was PCR amplified from its original construct hTERT-pGRN145 (ATCC, Manassas, Va.) and subcloned into pcDNA3.1 vector (Invitrogen, Grand Island, N.Y.). Briefly, normal mouse cortical astrocytes cells at p0 were transfected with 4 μg of non-linearized pcDNA 3.1 plasmid containing hTERT cDNA using Optifect reagent from Invitrogen. After overnight incubation, the transfection mixture was replaced with normal growth media. Selection of hTERT-expressing cells was then achieved by successive splitting bimonthly for 7 months in culture, thus eradicating cells that did not continue to divide in prolonged culture. In contrast to telomerase-negative control, which exhibited telomere shortening and senescence, telomerase-expressing clones (checked by western blot), divided continuously exceeding their normal life-span by at least 20 doublings. Later passages of these cell lines showed expression of astrocytic markers and maintained the youthful phenotype.

Human glioblastoma U87 cells were kindly donated by Alan LAST NAME and GL261 murine glioblastomas cells were obtained from the National Cancer Institute (Frederick, Md.). Cells were subcultured in DMEM, containing 10% Fetal Bovine Serum, and 1% penicillin-streptomycin and maintained at 37° C. with 95% air/5% CO2.

### 6.26 In-Vitro Model of the BBB

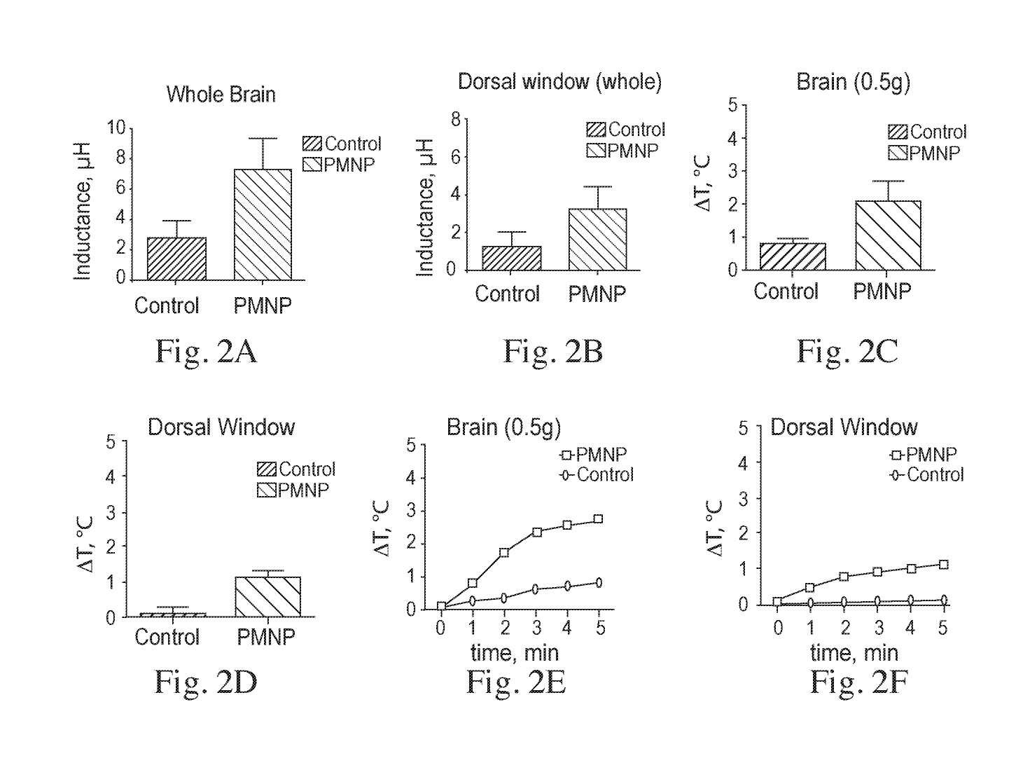

The in-vitro model of the BBB was prepared following a method established by Li et al. [15]. As shown in FIG. 2, cell culture inserts with a 0.4 μm porous membrane were flipped up-side down and coated with 100 μL, of a 30 μg/mL fibronectin (FN) solution in DPBS. The coated inserts were incubated for 1 hour at 37° C. with 95% air/5% CO₂ and IWCA were seeded at a density of 30,000 cells/cm². The cells were allowed to adhere to the FN coated porous membrane for 2 hours, after which they were flipped again and allowed to grow in the incubator for 2 days. bEnd.3 cells were then seeded on the luminal side of the insert at a density of 60,000 cells/cm². Upon endothelial confluence (4-6 days later) the diffusive permeability of the NPs was measured.

### 6.27 Immunostaining of Co-Culture

To label the cells in our model, the inserts were rinsed in PBS and fixed in 4% PFA for 15 minutes. The top side (endothelial) and the bottom side (astrocytic) were incubated with corresponding primary antibodies diluted in blocking solution (1×PBS, 0.4% Triton X-100, 10% BSA and 2% goat serums) at a ratio of 1:200. To mark endothelial cells we used a rabbit anti-VonWillebrand factor and to mark astrocytes we used mouse anti-GFAP. The inserts were then incubated with 488 anti-rabbit and 546 anti-mouse secondary antibodies diluted in blocking solution (1:1000). The porous membranes were carefully removed from the inserts using a razor blade and mounted on coverslips with mounting media containing DAPI for nuclear staining. Random images were taken with the LSM-510 Zeiss confocal microscope.

### 6.28 Characterization of the BBB In-Vitro Model

Endothelial monolayers only and co-cultures with astrocytes were characterized by measuring trans-endothelial electrical resistance (TEER) using electrode tweezers (EVOM-WPI) and by measuring permeability to 10 kDa Dextran following the method explained in the next section.

### 6.29 Permeability of the BBB Model

Inserts were rinsed twice with experimental medium, consisting of phenol red free DMEM supplemented with 1% BSA. The well containing the insert was filled with 1.5 mL of this medium and the luminal side of the insert was filled with 0.5 mL of either 10 kDa Dextran solution (10 mM) or NP solution (5 mg/ml) in experimental medium. An hour later, the medium in the well was mixed thoroughly by resuspending it at least 8 times, and 100 μL, were collected. This medium collection was repeated 2 more times. The samples collected were placed in a 96-well plate (Costar) and fluorescence was measured using a plate reader (BMG labtech, Ortenberg/Germany) with excitation/emission wavelengths of 485/520 nm. The standard curve (Fluorescence versus concentration) for Dextran and NPs was used to convert all fluorescence measurements into concentrations. The concentration of Dextran or NP versus time for the three time points was plotted and the slope was used to calculate the permeability of the BBB model by using the following equation:

P_{d}=([dC/dt]*V)/(A*Co)

Where

[dC/dt]: is the slope of the concentration versus time curve.

V: is the average volume in the lower compartment (1.35 cm³)

A: area of insert membrane (0.9 cm²)

Co: Initial concentration on top (luminal)

The permeability of the PMNPs was tested in the same way but during an interval of two hours, taking samples every 30 minutes and one extra sample taken the next day. In parallel experiments, a magnetic field was placed below the tray holding the inserts to determine if a magnetic field increased the flux of PMNPs across the BBB model. The PMNP luminal concentration was 0.25 mg/mL.

### 6.30 Nanoparticle Tracking Across the Endothelium

To track the NPs in the in-vitro model, once the permeability experiments were run, the co-cultures were rinsed 3 times with PBS to remove excess solution of NPs on the luminal side and fixed in 4% PFA for 15 minutes. The endothelial side was incubated overnight with CellTracker DiI previously reconstituted in DMSO as per manufacturer's instructions and then diluted 1:400 in PBS. The porous membranes were removed from the insert and mounted on coverslips with a mounting media containing DAPI for nuclear staining. Several images were taken with the LSM-510 Zeiss confocal microscope.

### 6.31 Cell Uptake

Uptake of PMNPs by U87 cells was confirmed by binding an mCherry plasmid to the nanoparticles. A 10 mg/mL PMNP solution was mixed and bound with/to an mCherry plasmid solution (20 μg/mL). U87 Cells were plated on 35 mm Ibidi imaging dishes and grown for two days with 10% DMEM. The cells were then incubated with 1.5 μL of mCherry plasmid-PMNP solution in 1 mL of Optimem. The final concentration of plasmid was 30 pg/mL, corresponding to a 15 ng/mL PMNP solution. After four hours, control cells (without the mCherry-PMNP solution) and experimental cells were washed with PBS and imaged with a 40× dry objective using the same optical variables.

### 6.32 Cell Viability Assay

Cells were plated in 24-well trays and allowed to become confluent then incubated with naked curcumin or different curcumin NP concentrations for 48 h. Cell viability was quantified using Presto Blue (Invitrogen) an assay that contains a cell permeable resazurin-based solution that is modified by the reducing environment of the viable cell becoming highly fluorescent. Using a plate reader, cell viability was quantified after 0.5-1 hours of incubation with Presto Blue. All measurements were always accompanied by control wells and by calibration wells to account for background fluorescence using the same culture media and the same NP concentrations without the cells.

### 6.33 In Vivo Brain Tumor Model

The procedures conducted herein were in full compliance with the Institutional Animal Care and Use Committee (IACUC). Briefly, C57BL/6 mice were anesthetized with an oxygen isoflurane mixture. Once hind paw retraction reflex was null, the head was shaved and disinfected with 70% v/v alcohol. A small circular slit was punctured in the skull approximately 3 mm above and to the right of the bregma, where 0.2 μL of GL261 cell pellet was introduced in the brain with a microliter syringe (Hamilton). The syringe needle was removed after at least five seconds post injection in order to avoid cell suspension reflux.

### 6.34 In Vivo and Ex-Vivo Localization of PMNPs

Two weeks after the GL261 tumor cells were introduced in the brain of C57BL/6 mice, a solution of 1.5 mg of Rhodamine tagged PMNPs was injected into the mice via tail vein to observe whether PMNP localization to the tumor could be enhanced by using a magnetic field placed on the head, close to the tumor site. The localization of the PMNPs was observed using a whole animal fluorescence imaging system (Carestream). The animals were anesthetized using an oxygen/isothesia mix and placed in the imaging compartment. The pictures before and after treatment were taken and compared. Control mice (with tumors but without PMNPs injected) were compared to mice injected with PMNPs that were either exposed or not for 45-60 minutes to the action of a magnetic field. One of the animals was injected with GFP-GL261 instead to have a better image of PMNPs localization. This animal was injected with the PMNPs and exposed to the action of the magnetic field, its brain was extracted and cut in about 1 mm slices and placed on coverslips for quick observation under the microscope. We avoided fixing the brain with paraformaldehyde to avoid tissue autofluorescence and to be able to distinguish clearly the presence of green fluorescent tumor cells from regular tissue. The tumor cells were localized first and then the wavelength was changed to look for the Rhodamine tagged PMNPs in the same field.

### 6.35 Statistical Analysis

Permeability and cell viability measurements are presented as means±SE. When applicable, all comparisons and statistics were performed in Excel, using the Student's t-test. For all of the experiments, P<0.05 was considered significant.

### 6.36 Results

### TEER and Permeability to 10 kDa Dextran of Endothelium Versus Co-Culture BBB Model

Co-cultures and endothelial monolayers were characterized by measuring TEER and permeability to 10 kDa Dextran. The results show that barrier function of the endothelium is significantly increased by coculturing astrocytes on the opposite side of the insert (p-value<0.05). Endothelium versus co-culture values are as follow: TEER [Ω cm²]: 18.97±1.41 Vs. 28.23±1.7 n=12 and Pe (X 10⁻⁶ cm/s): 4.02±0.59 Vs. 2.44E-6±0.24 n=9. Based on these results demonstrating an almost two fold higher resistance and lower permeability in the cocultures compared to endothelium alone, we measured NP permeability in the co-culture system because it resembles more closely the tightness of the BBB.

Standard Curves for the NPs

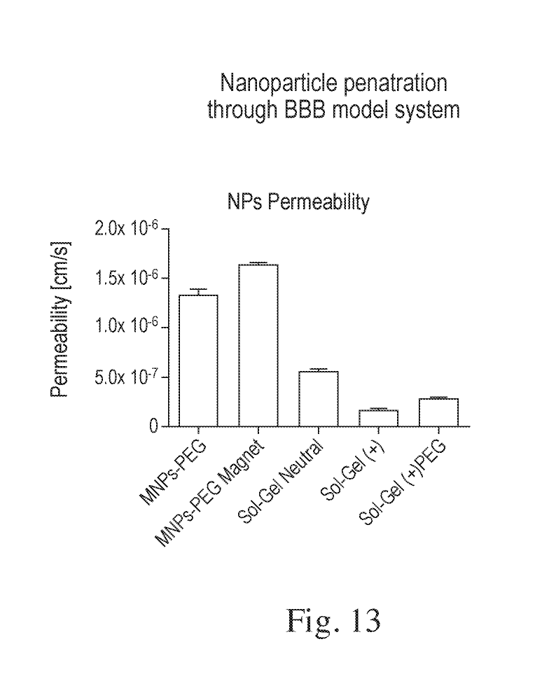

To measure NP permeability across the co-cultures we first run standard curves to relate concentration of the NPs to fluorescence intensity. FIG. 13 shows standard curves for each of the nanoparticles. In all cases the relationship between nanoparticle concentration and fluorescent intensity was linear (R²≧0.98). We tested NPs fabricated with different characteristics; PMNPs coated with PEG (PMNP-PEG), Sol-Gel-NPS with neutral charge, positive charge (+) and positive plus PEG (+)-PEG. PMNPs show higher fluorescence per gram of nanoparticles than the Sol-Gel based NPs.

Nanoparticle Permeability of the BBB Model

As shown in FIG. 13, the permeability (Pe) of the endothelial/astrocyte cocultures to PMNPs coated with PEG (PMNP-PEG) was found to be significantly higher than that to positively charged (+)Sol-Gel and neutral NPs (p-values=1.4E-6, and 2.5E-5 respectively). Positively charged nanoparticles were least permeable but permeability was significantly increased by PEGylation ((+)-PEG, p-value=2.2E-3). In addition, we observed that placing a magnetic field below the abluminal compartment significantly increased the flux of PMNPs-PEG (p-value=2.0E-3), suggesting that an innovative approach could be developed by directing PMNPS to a specific location via a magnetic field. Based on these results, priority may be given to paramagnetic NPs for animal studies due to their superior performance. In a separate set of experiments, we varied the methanol percentage during the Sol-Gel NP synthesis (47%, 55%, 63% and 71%), but we did not observe any significant change in permeability (data not shown). We confirmed that neutral NPs had higher permeability values than positively charged ones.

Cell Viability of Curcumin Versus Curcumin Loaded NPs

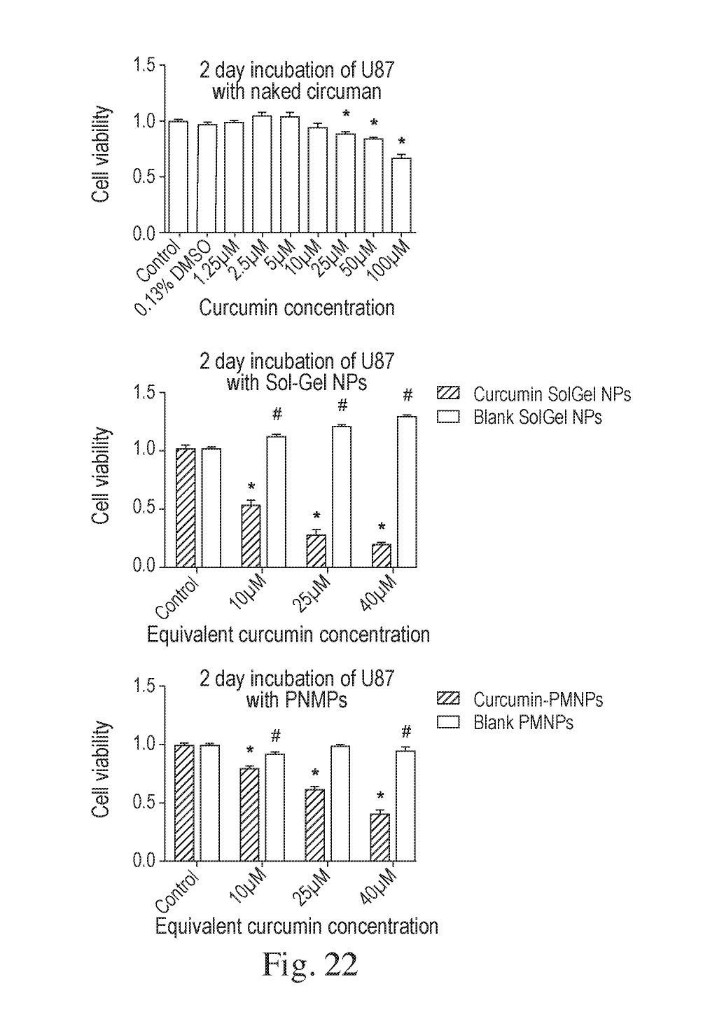

U-87 used as an vitro model of human glioblastoma were incubated with either Curcumin or Curcumin loaded NPs. FIG. 22 shows that the mass of Curcumin needed to reduce cell viability was decreased when encapsulated in the nanoparticles. The best performance was achieved by Curcumin loaded PMNPs where an equivalent of only 0.625 uM Curcumin was able to significantly decrease cell viability by 20%; equivalent reduction in U87 cell viability required 5 uM naked curcumin.

In-Vivo and Ex-Vivo Localization of the PMNPs.

Once injected, the Rhodamine-PMNPs possessed enough signal strength to localize them inside the mice and within the capabilities of the imager used. Mice that were exposed for 45-60 minutes to the action of a magnetic field show a significant increase in fluorescent signal at the tumor site. To further confirm the result, one of the mice was injected with GFP-GL261 cells which formed a fluorescent tumor. The animal was then injected with the Rhodamine-PMNPs and a magnetic field placed in the vicinity of the tumor as in the previous experiments. After treatment the brain was excised to observe for the presence of Rhodamine-PMNPs. These PMNPs are localized inside the tumor region.

All publications mentioned herein are hereby incorporated in their entireties into the subject application. Where there is an apparent conflict between a term as used herein and the same term as used in a publication incorporated by reference herein, the present specification is understood to provide the controlling definition.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### REFERENCES

1. Araujo J, Logothetis C: Dasatinib: a potent SRC inhibitor in clinical development for the treatment of solid tumors, Cancer Treat Rev 2010, 36:492-500
2. Schmid M C, Varner J A: Myeloid cells in the tumor microenvironment: modulation of tumor angiogenesis and tumor inflammation, J Oncol 2010, 2010:201026
3. Lin E Y, Pollard J W: Tumor-associated macrophages press the angiogenic switch in breast cancer, Cancer Res 2007, 67:5064-5066
4. Lin E Y, Li J F, Gnatovskiy L, Deng Y, Zhu L, Grzesik D A, Qian H, Xue X N, Pollard J W: Macrophages regulate the angiogenic switch in a mouse model of breast cancer, Cancer Res 2006, 66:11238-11246
5. Lin E Y, Li J-F, Bricard G, Wang W, Deng Y, Sellers R, Porcelli S A, Pollard J W: VEGF restores delayed tumor progression in tumors depleted of macrophages, Molecular Oncology, 2007, 1:288
6. Lin E Y, Nguyen A V, Russell R G, Pollard J W: Colony-stimulating factor 1 promotes progression of mammary tumors to malignancy, J Exp Med 2001, 193:727-740.
7. Solinas G, Germano G, Mantovani A, Allavena P: Tumor-associated macrophages (TAM) as major players of the cancer-related inflammation, J Leukoc Biol 2009, 86:1065-1073
8. Kozlow W, Guise T A: Breast cancer metastasis to bone: mechanisms of osteolysis and implications for therapy, J Mammary Gland Biol Neoplasia 2005, 10:169-180
9. Chirgwin J M, Guise T A: Skeletal metastases: decreasing tumor burden by targeting the bone microenvironment, J Cell Biochem 2007, 102:1333-1342
10. Roodman G D: Mechanisms of bone metastasis, N Engl J Med 2004, 350:1655-1664
11. Mizutani K, Sud S, McGregor N A, Martinovski G, Rice B T, Craig M J, Varsos Z S, Roca H, Pienta K J: The chemokine CCL2 increases prostate tumor growth and bone metastasis through macrophage and osteoclast recruitment, Neoplasia 2009, 11:1235-1242
12. Zhang J, Lu Y, Pienta K J: Multiple roles of chemokine (C—C motif) ligand 2 in promoting prostate cancer growth, J Natl Cancer Inst 2010, 102:522-528
13. Morgan G, Lipton A: Antitumor effects and anticancer applications of bisphosphonates, Semin Oncol 2010, 37 Suppl 2:S30-40
14. Pavlakis N, Schmidt R, Stockler M: Bisphosphonates for breast cancer, Cochrane Database Syst Rev 2005, CD003474
15. Ruggiero S L, Mehrotra B, Rosenberg T J, Engroff S L: Osteonecrosis of the jaws associated with the use of bisphosphonates: a review of 63 cases, J Oral Maxillofac Surg 2004, 62:527-534
16. Woo S B, Hellstein J W, Kalmar J R: Narrative [corrected] review: bisphosphonates and osteonecrosis of the jaws, Ann Intern Med 2006, 144:753-761
17. Lin E Y, Orlofsky A, Berger M S, Prystowsky M B: Characterization of A1, a novel hemopoietic-specific early-response gene with sequence similarity to bcl-2, J Immunol 1993, 151:1979-1988
18. Lin E Y, Orlofsky A, Wang H G, Reed J C, Prystowsky M B: A1, a Bcl-2 family member, prolongs cell survival and permits myeloid differentiation, Blood 1996, 87:983-992
19. Park S I, Soki F N, McCauley L K: Roles of Bone Marrow Cells in Skeletal Metastases: No Longer Bystanders, Cancer Microenviron 2011,
20. Lin E Y, Gouon-Evans V, Nguyen A V, Pollard J W: The macrophage growth factor, CSF-1, in mammary gland development and cancer, Journal of Mammary Gland Development and Neoplasia 2002, 7:147-162
21. Conway J G, McDonald B, Parham J, Keith B, Rusnak D W, Shaw E, Jansen M, Lin P, Payne A, Crosby R M, Johnson J H, Frick L, Lin M H, Depee S, Tadepalli S, Votta B, James I, Fuller K, Chambers T J, Kull F C, Chamberlain S D, Hutchins J T: Inhibition of colony-stimulating-factor-1 signaling in vivo with the orally bioavailable cFMS kinase inhibitor GW2580, Proc Natl Acad Sci USA 2005, 102:16078-16083
22. Irvine K M, Burns C J, Wilks A F, Su S, Hume D A, Sweet M J: A CSF-1 receptor kinase inhibitor targets effector functions and inhibits pro-inflammatory cytokine production from murine macrophage populations, Faseb J 2006, 20:1921-1923
23. Ahren M, Selegard L, Klasson A, Soderlind F, Abrikossova N, Skoglund C, Bengtsson T, Engstrom M, Kall P O, Uvdal K: Synthesis and characterization of PEGylated Gd2O3 nanoparticles for MRI contrast enhancement, Langmuir 2010, 26:5753-5762
24. Auletta C S: Acute, subchronic, and chronic toxicology. Edited by Derelanko M J, Hollinger M A. Boca Raton, London, New York, Washington D.C., CRC Press, 2002, p.pp. 69-126

1. A method of making modified PMNP comprising the steps of:
(i) adding fatty acid to PMNP core to form a mixture;
(ii) Sonicating the mixture;
(iii) Spinning the sonicated mixture and washing in deionized water;
(iv) Drying and lyophilizing the washed mixture to form a powder;
(v) mixing the lyophilized powder with a non-aqueous concentrated solution of a therapeutic agent to form a mixture;
(vi) sonicating the mixture from step (v);
(vii) spinning the sonicated mixture and washing in deionized water.

2. A method of making a modified PMNP comprising the steps of:
(i) adding fatty acid to PMNP core to form a mixture;
(ii) Sonicating the mixture;
(iii) Spinning the sonicated mixture and washing in deionized water to form an aqueous suspension;
(iv) mixing the aqueous suspension with a non-aqueous concentrated solution comprising a therapeutic agent;
(v) sonicating the mixture from step (vi);
(vi) removing the non-aqueous solvent.

3. The method of claim 1 wherein the therapeutic agent is Adriamycin, taxol, curcumin, dasationib, melanin, allosteric effector, albumin, plasmid, siRNA or a combination thereof.

4. A method of making a drug-loaded albumin-coated paramagnetic nanoparticle (alb-PMNP) comprising the steps of:
(i) mixing an ethanol in methanol solution comprising PMNP core with a methanol solution comprising a therapeutic agent to form a mixture;
(ii) sonicating the mixture;
(iii) adding an aqueous solution comprising albumin to the sonicated mixture.

5. The method of claim 4 further comprising the step of:
(i) adding methoxy PEG-DSPE, fluorescence-labeled PEG-DSPE, or a derivatized PEG-DSP to the sonicated mixture in step (iii).

6. The method of claim 5 wherein the PEG-DSPE comprises a reactive species including maleimide, amine, thiol or a combination thereof.

7. The method of claim 6 wherein the reactive species is attached to a fluorophore, PET imaging agent, peptide, antibody, aptamer, contrasting agent or a combination thereof.

8. The method of claim 7 wherein the peptide is CXCR4 antagonistic peptide.

9. A method of making modified PMNP comprising the steps of:
(i) adding 3-mercaptopropyl-trimethoxysilane (3MPTS) or (N-(2-Aminoethoxyl)-11-Aminoundecyl trimethoxysilane) (APTS) to a solution containing PMNP core in deionized water to form a mixture;
(ii) Sonicating the mixture in step (i);
(iv) incubating the sonicated mixture at 4° C.;
(v) washing the mixture in deionized water; and
(vi) adding 4′-dithiodipyridine (4-PDS) to form a mixture.

10. The method of claim 9 further comprising the step of:
(i) adding 2-imminothiolane & mal-PEG-5K to the mixture of step (vi).

11. The method of claim 10 further comprising the step of:
(i) adding dithiothreitol (DTT) to the mixture of step (vi);
(ii) treating the mixture with buffer saturated with pure NO gas.

12. The method of claim 1 wherein the PMNP core comprises substantially of Gd₂O₃ or iron oxide.

13. The method of claim 12 wherein the Gd₂O₃ is doped with europium or other lanthanides.

14. The method of claim 1 wherein the fatty acid is oleic acid.

15. A method of delivering the modified PMNP to a target location in a subject comprising:
(i) administering to the subject an effective amount of the modified PMNP prepared by the method of claim 1;
(ii) applying a magnetic field to the subject, such that the magnetic field is present at the target location at a strength sufficient to attract the modified PMNP.

16. The method of claim 15 wherein the modified PMNP is administered systemically.

17. The method of claim 15 wherein the location of the modified PMNP is monitored using MRI.

18. The method of claim 15 wherein the modified PMNP comprises fluorophores.

19. A method of treating cancer in a subject comprising: (i) administering to the subject an effective amount of the modified PMNP prepared by the method of claim 1; and (ii) applying a magnetic field to the subject at the location of the cancer, and wherein the magnetic field is at a strength sufficient to attract the modified PMNP to the cancer.

20. The method of claim 19 wherein the therapeutic agent is a chemotherapeutic drug, small organic molecule, cytotoxic drug, or a combination thereof.

21. The method of claim 19 wherein the cancer is pancreatic cancer, CNS cancer, bone cancer, hypoxic tumor.

22. The method of claim 19 wherein the subject is treated with a second cancer therapy.

23. A method of treating cancer in a subject comprising: (i) administering to the subject an effective amount of the modified PMNP prepared by the method of claim 4; and (ii) applying a magnetic field to the subject at the location of the cancer, and wherein the magnetic field is at a strength sufficient to attract the modified PMNP to the cancer.

24. The method of claim 23 wherein the peptide binds to a cell-surface target.

25. A method of treating sickle cell disease in a subject comprising administering to the subject an effective amount of the modified PMNP prepared by the method of claim 1, wherein the therapeutic agent is an allosteric effector.

26. A method of treating an inflammation in a subject comprising: (i) administering to the subject an effective amount of the modified PMNP prepared by the method of claim 1; and (ii) applying a magnetic field to the subject at the location of the inflammation, and wherein the magnetic field is at a strength sufficient to attract the modified PMNP to the predetermined location.

27. The method of claim 26 wherein the inflammation is at a joint.

28. A method of treating or reducing a reperfusion injury or ischemia in a subject comprising: (i) administering to the subject an effective amount of the modified PMNP prepared by the method of claim 1; and (ii) applying a magnetic field to the subject at the location of the reperfusion injury or ischemia, and wherein the magnetic field is at a strength sufficient to attract the modified PMNP to the reperfusion injury or ischemia.

29. A method of imaging a predetermined location in a subject comprising:
(i) administering to the subject an effective amount of the modified PMNP prepared by the method of claim 1; (ii) applying a magnetic field to the subject predetermined location at a strength sufficient to attract the modified PMNP to the predetermined location; and (iii) collecting an imaging signal from the predetermined location using an imaging device so as to thereby image the predetermined location.

30. A method of increasing oxygen levels in a target tissue in a subject having a disorder comprising: (i) administering to the subject an effective amount of the modified PMNP prepared by the method of claim 1; and (ii) applying a magnetic field to the subject at the predetermined location where an increased oxygen level is desired, and wherein the magnetic field is at a strength sufficient to attract the modified PMNP to the predetermined location.

31. The method of claim 30 wherein the disorder is cancer, hypoxic tumor, sickle cell anemia, or local hypoxic conditions.

32. A modified paramagnetic nanoparticle (PMNP) comprising a PMNP core, which core comprises a coating, said coating comprising oleic acid, a fatty acid, albumin, or a combination thereof, said coating is dispersed therewith an allosteric effector of hemoglobin, curcumin, melanin, siRNA, plasmids, nitro fatty acids, adriamycin, taxol, or a combination thereof and wherein polymer PEG-DSPE are attached to the PMNP core.

33. The modified PMNP of claim 32 wherein the allosteric effector of hemoglobin is, 2, 3-Bisphosphoglycerate (2, 3-BPG), Myo-inositol trispyrophosphate (ITPP), or a combination thereof.

34. The modified PMNP of claim 32 wherein the coating comprises an albumin and wherein the coating is dispersed therewith curcumin, Adriamycin, taxol and wherein the polymer 1, 2-distearoyl-sn-glycero-3-phosphoethanolamine-N-[amino(polyethylene glycol)-2000 (PEG-DSPE) is attached to the PMNP core.

35. The modified PMNP of claim 34 wherein the PEG-DSPE comprises a reactive species including maleimide, amine, thiol or a combination thereof.

36. The modified PMNP of claim 35 wherein the reactive species is attached to fluorophores, PET imaging agents, peptides, antibodies, aptamers, contrasting agents or a combination thereof.

37. The modified PMNP of claim 32 wherein the coating comprises oleic acid and wherein the coating is dispersed therewith curcumin, Adriamycin, taxol and wherein the polymer 1, 2-distearoyl-sn-glycero-3-phosphoethanolamine-N-[amino(polyethylene glycol)-2000 (PEG-DSPE) is attached to the PMNP core.

38. The modified PMNP of claim 37 wherein the PEG-DSPE comprises a reactive species including maleimide, amine, thiol or a combination thereof.

39. The modified PMNP of claim 38 wherein the reactive species is attached to fluorophores, PET imaging agents, peptides, antibodies, aptamers, contrasting agents or a combination thereof.

40. The method of claim 1 further comprising the step of:
(i) adding methoxy PEG-DSPE, fluorescence-labeled PEG-DSPE, or a derivatized PEG-DSP to the sonicated mixture in step (iii).

41. The method of claim 40 wherein the PEG-DSPE comprises a reactive species including maleimide, amine, thiol or a combination thereof.

42. The method of claim 41 wherein the reactive species is attached to a fluorophore, PET imaging agent, peptide, antibody, aptamer, contrasting agent or a combination thereof.

43. The method of claim 42 wherein the peptide is CXCR4 antagonistic peptide.
